(19) United States
(12) Patent Application Publication
(10) Pub. No.: US 2024/0252497 A1
(43) Pub. Date: Aug. 1, 2024

(54) USE OF COMPLEMENT FACTOR D INHIBITOR FOR TREATMENT OF GEOGRAPHIC ATROPHY SECONDARY TO AGE-RELATED MACULAR DEGENERATION

(71) Applicant: Alexion Pharmaceuticals, Inc., Boston, MA (US)

(72) Inventors: Aleksandar Skuban, New Hope, PA (US); Mingjun Huang, New Haven, CT (US); David Boyer, New Haven, CT (US); Jose Rivera, Kensington, CT (US); Ya-Ping Ko, Cheshire, CT (US); Xiang Gao, Guilford, CT (US)

(73) Assignee: Alexion Pharmaceuticals, Inc., Boston, MA (US)

(21) Appl. No.: 18/564,202

(22) PCT Filed: May 26, 2022
(86) PCT No.: PCT/US2022/031200

§ 371 Date: Nov. 27, 2023

Related U.S. Application Data
(60) Provisional application No. 63/194,523, filed on May 28, 2021.

(51) Int. Cl. A61K 31/506; A61P 27/02

(52) U.S. Cl. CPC A61K 31/506; A61P 27/02

(57) ABSTRACT

Disclosed herein are methods for treating geographic atrophy secondary to age-related macular degeneration (AMD) and intermediate AMD in a subject. The methods include administering to the subject a therapeutically effective amount of a small molecule complement factor D inhibitor.

### CROSS-REFERENCE TO RELATED APPLICATION

This application claims the benefit of priority to U.S. Application No. 63/194,523, filed May 28, 2021, which is incorporated by reference herein for all purposes.

### BACKGROUND

The complement system is a key component of innate immunity. It consists of a large group of plasma and membrane bound proteins that play a central role in the defense against infection and in the modulation of immune and inflammatory responses. The complement system can be activated via three distinct pathways, namely, the classical, the alternative and the lectin pathways. Complement activation triggers a sequence of biological reactions. The classical pathway can be activated by immune complexes or by substances such as C-reactive protein, and the complement components involved include C1, C2, C4 and C3. The alternative pathway provides a rapid, antibody-independent route of complement activation and amplification. The alternative pathway directly activates C3 when it interacts with certain activating surfaces (e.g., zymosan, lipopolysaccharides) and involves the actions of C3, Factor B, Factor D, and properdin. The activation of the lectin pathway is also independent of immune complex generation, and can be achieved by interaction of certain serum lectins, such as mannose binding lectin (MBL), with mannose and N-acetyl glucosamine residues present in abundance in bacterial cell walls.

In the normal eye, the complement system is continuously activated at low levels and both membrane-bound and soluble intraocular complement regulatory proteins tightly regulate this spontaneous complement activation. This allows protection against pathogens without causing any damage to self-tissue and vision loss. Activated complement, however, has the potential to inflict damage to self-tissue. The presence and activation of complement has been suggested to play a crucial role in the pathogenesis of a large number of diseases, including ocular diseases (See, e.g., Thurman et al., J. Immunol. 2006; 176:1305-1310).

The alternative complement system has been implicated in a large number of ocular disorders that may affect the anterior region of the eye, the posterior region of the eye or the whole eye. One notable ocular disorder in which complement has been implicated is age-related macular degeneration (AMD), which is a leading cause of vision loss in industrialized countries. There are two main types of AMD; the dry (non-neovascular) form and the wet (neovascular) form. Geographic atrophy (GA) is a chronic progressive degeneration of the macula, and is considered as part of late-stage age-related macular degeneration (AMD). The condition leads to central scotomas and permanent loss of visual acuity. The disease is characterized by localized, sharply demarcated atrophy of outer retinal tissue, retinal pigment epithelium and choriocapillaris.

Multiple agents with different mechanism of actions, including neuroprotectors, immunomodulators, anti-inflammatory agents, and complement inhibitors have been investigated or are under clinical investigation for the treatment of GA (see Nebbioso et al., Int J Mol Sci. 2019; 20(7); and Sastre-Ibáñez et al., Arch Soc Esp Oftalmol. 2018; 93(1):22-34). However, no treatment is currently approved for this indication, underlining a substantial unmet medical need for patients with GA.

It is an object of the present disclosure to provide new treatment regimens for AMD, particularly GA secondary to AMD.

### SUMMARY OF THE DISCLOSURE

Provided herein are methods for treating age-related macular degeneration (AMD), particularly geographic atrophy (GA) secondary to AMD, in a subject with a small molecule complement factor D (CFD) inhibitor. The present disclosure is based, in part, on the use of oral CFD inhibitors, such as Compound 1 or a pharmaceutically acceptable salt thereof, as a first-in-class treatment option for patients with GA secondary to AMD.

As an orally administered small molecule, Compound 1 is advantageous over therapeutic agents being investigated for the use in treating GA, which rely in intravitreal administration. In this regard, oral CFD inhibitors, such as Compound 1, not only provide GA patients with a more convenient and accessible option for therapy, but also help reduce the patient and caregiver burden for by lowering the need for clinical visits and minimize the risk of ocular injection-related adverse events. Further, the inventors have discovered that oral administration for Compound 1 resulted in systemic exposure and enabled concurrent treatment of both eyes and also offered the advantage of localization and retention at the target tissues, which is, in part, due to the ability of Compound 1 to cross the blood-retina barrier and target, with specific affinity, for melanin-containing tissues. In this manner, Compound 1 of the present disclosure offers at least two potential advantages over other drugs in development: (a) oral administration of Compound 1 results in its selective retention in ocular tissues containing melanin (e.g., choroid and retinal pigment epithelium (RPE)) as well as offering direct delivery to the retina and (b) systemic administration of Compound 1 effectively targets both eyes, which is advantageous over therapies that target the diseased eye, e.g., intravitreal injection. Given the overall pharmaceutical profile of Compound 1, the present disclosure provides that a treatment regimen with Compound 1 leads to better patient compliance and improved clinical outcomes in patients with GA.

In a first aspect, the present disclosure features a method of treatment, which includes treating GA secondary to AMD in at least one eye in a subject, said treating including administering to the subject an effective amount of (2S,4R)-1-(2-(3-acetyl-5-(2-methylpyrimidin-5-yl)-1H-indazol-1-yl)acetyl)-N-(6-bromopyridin-2-yl)-4-fluoropyrrolidine-2-carboxamide (Compound 1; also known as ALXN2040 and danicopan):

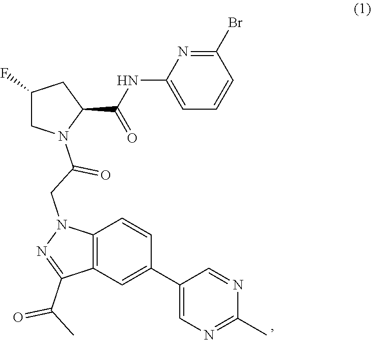 [SMILES: CC(=O)c1nn(CC(=O)N2C[C@H](F)C[C@H]2C(=O)Nc2cccc(Br)n2)c2ccc(-c3cnc(C)nc3)cc12]

or a pharmaceutically acceptable salt thereof. In some embodiments, the treatment includes slowing or reversing a progression of the GA, wherein the slowing the progression of the GA includes a reduction in a mean rate of increase in a total GA lesion area from baseline in the at least one eye (e.g., as measured by fundus autofluorescence [FAF]), and the reversing the progression of the GA includes decreasing the total GA lesion area from baseline (e.g., increasing a mean rate of decrease in the total GA lesion area) in the at least one eye (e.g., as measured by fundus autofluorescence [FAF]).

In some embodiments, Compound 1 or the pharmaceutically acceptable salt thereof is administered in a dosing regimen of about 50-250 mg twice daily (BID), e.g., about 100 mg BID or about 200 mg BID.

In some embodiments, Compound 1 or the pharmaceutically acceptable salt thereof is administered in a dosing regimen of about 200 mg to about 800 mg once daily (QD), e.g., about 400 mg QD.

In some embodiments, Compound 1 or the pharmaceutically acceptable salt thereof is systemically administered (e.g., orally or subcutaneously administered). In some embodiments, Compound 1 or the pharmaceutically acceptable salt thereof is ophthalmically administered.

In some embodiments, the subject is at least about 60 years of age (e.g., at least 65 years of age, at least about 70 years of age, at least about 75 years of age, or at least about 80 years of age).

In some embodiments, effective treatment (e.g., slowing or reversing the progression of the GA) is observed within 104 weeks of treatment (e.g., within 78 weeks of treatment, within 52 weeks of treatment, or within 26 weeks of treatment).

In some embodiments, the GA secondary to AMD includes loss of retinal pigment epithelium (RPE) in the eye, optionally progressing to loss of center of macula (e.g., fovea) of the eye.

In some embodiments, the at least one eye has a GA lesion that is completely visualized on a macula centered color fundus photography (CFP) image and is imaged in its entirety.

In some embodiments, the at least one eye has a GA area of 0.35 to 17.76 mm² (e.g., 0.5 to 17.76 mm²) as measured by FAF.

In some embodiments, the at least one eye does not have exudative neovascular age-related macular degeneration (nAMD).

In some embodiments, the subject has nAMD in the at least one eye.

In some embodiments, the slowing of the progression of the GA further includes a reduction in (1) a mean rate of increase (by about 0.1 mm/year, about 0.2 mm/year, about 0.3 mm/year, about 0.4 mm/year, about 0.5 mm/year, about 0.6 mm/year, about 0.7 mm/year, about 0.8 mm/year, about 0.9 mm/year, about 1 mm/year, about 1.1 mm/year, about 1.2 mm/year, about 1.3 mm/year, about 1.4 mm/year, about 1.5 mm/year, about 1.6 mm/year, about 1.7 mm/year, about 1.8 mm/year, about 1.9 mm/year, about 2 mm/year, about 2.1 mm/year, about 2.2 mm/year, about 2.3 mm/year, about 2.4 mm/year, about 2.5 mm/year, about 2.6 mm/year, about 2.7 mm/year, or about 2.8 mm/year) in a square root of a total GA lesion area from baseline as measured by FAF in (a) the at least one eye; (b) a fellow eye if GA is present at the fellow eye at baseline; or (c) both the at least one eye and the fellow eye, regardless of baseline GA status of the at least one eye and the fellow eye; and the reversing the GA further includes a mean decrease (e.g., by about 0.1 mm, about 0.2 mm, about 0.3 mm, about 0.4 mm, about 0.5 mm, about 0.6 mm, about 0.7 mm, about 0.8 mm, about 0.9 mm, about 1 mm, about 1.1 mm, about 1.2 mm, about 1.3 mm, about 1.4 mm, about 1.5 mm, about 1.6 mm, about 1.7 mm, about 1.8 mm, about 1.9 mm, about 2 mm, about 2.1 mm, about 2.2 mm, about 2.3 mm, about 2.4 mm, about 2.5 mm, about 2.6 m, about 2.7 mm, or about 2.8 mm) in the square root of the total GA lesion area (e.g., an increase in a mean rate of decrease in the square root of the total GA lesion area, e.g., by about 0.1 mm/year, about 0.2 mm/year, about 0.3 mm/year, about 0.4 mm/year, about 0.5 mm/year, about 0.6 mm/year, about 0.7 mm/year, about 0.8 mm/year, about 0.9 mm/year, about 1 mm/year, about 1.1 mm/year, about 1.2 mm/year, about 1.3 mm/year, about 1.4 mm/year, about 1.5 mm/year, about 1.6 mm/year, about 1.7 mm/year, about 1.8 mm/year, about 1.9 mm/year, about 2 mm/year, about 2.1 mm/year, about 2.2 mm/year, about 2.3 mm/year, about 2.4 mm/year, about 2.5 mm/year, about 2.6 mm/year, about 2.7 mm/year, or about 2.8 mm/year) from baseline as measured by FAF in (a) the at least one eye; (b) the fellow eye if GA is present at the fellow eye at baseline; or (c) both the at least one eye and the fellow eye, regardless of baseline GA status in the at least one eye and the fellow eye.

In some embodiments, the slowing of the progression of the GA further includes a reduction in (2) a mean rate of increase in a total GA lesion area (by about 0.01 mm²/year, about 0.05 mm²/year, about 0.1 mm²/year, about 0.15 mm²/year, about 0.2 mm²/year, about 0.3 mm²/year, about 0.4 mm²/year, about 0.5 mm²/year, about 0.6 mm²/year, about 0.7 mm²/year, about 0.8 mm²/year, about 0.9 mm²/year, about 1 mm²/year, about 1.1 mm²/year, about 1.2 mm²/year, about 1.3 mm²/year, about 1.4 mm²/year, about 1.5 mm²/year, about 1.6 mm²/year, about 1.7 mm²/year, about 1.8 mm²/year, about 1.9 mm²/year, about 2 mm²/year, about 2.1 mm²/year, about 2.2 mm²/year, about 2.3 mm²/year, about 2.4 mm²/year, about 2.5 mm²/year, about 2.6 mm²/year, about 2.7 mm²/year, about 2.8 mm²/year, about 2.9 mm²/year, about 3.0 mm²/year, about 3.5 mm²/year, about 4 mm²/year, about 4.5 mm²/year, about 5 mm²/year, about 5.5 mm²/year, about 6 mm²/year, about 6.5 mm²/year, about 7 mm²/year, about 7.5 mm²/year, or about 8.0 mm²/year) from baseline as measured by FAF in (a) the at least one eye; (b) a fellow eye if GA is present at the fellow eye at baseline; or (c) both the at least one eye and the fellow eye, regardless of baseline GA status; and the reversing of the progression of the GA further includes a mean decrease (by about 0.01 mm², about 0.05 mm², about 0.1 mm², about 0.15 mm², about 0.2 mm², about 0.3 mm², about 0.4 mm², about 0.5 mm², about 0.6 mm² about 0.7 mm², about 0.8 mm², about 0.9 mm², about 1 mm², about 1.1 mm², about 1.2 mm², about 1.3 mm², about 1.4 mm², about 1.5 mm², about 1.6 mm², about 1.7 mm², about 1.8 mm², about 1.9 mm², about 2 mm², about 2.1 mm², about 2.2 mm², about 2.3 mm², about 2.4 mm², about 2.5 mm², about 2.6 mm², about 2.7 mm², about 2.8 mm², about 2.9 mm², about 3.0 mm², about 3.5 mm², about 4 mm², about 4.5 mm², about 5 mm², about 5.5 mm², about 6 mm², about 6.5 mm², about 7 mm², about 7.5 mm², or about 8.0 mm²) in the total GA lesion area (e.g., an increase in a mean rate of decrease in the total GA lesion area, e.g., by about 0.01 mm²/year, about 0.05 mm²/year, about 0.1 mm²/year, about 0.15 mm²/year, about 0.2 mm²/year, about 0.3 mm²/year, about 0.4 mm²/year, about 0.5 mm²/year, about 0.6 mm²/year, about 0.7 mm²/year, about 0.8 mm²/year, about 0.9 mm²/year, about 1 mm²/year, about 1.1 mm²/year, about 1.2 mm²/year, about 1.3 mm²/year, about 1.4 mm²/year, about 1.5 mm²/year, about 1.6 mm²/year, about 1.7 mm²/year, about 1.8 mm²/year, about 1.9 mm²/year, about 2 mm²/year, about 2.1 mm²/year, about 2.2 mm²/year, about 2.3 mm²/year, about 2.4 mm²/year, about 2.5 mm²/year, about 2.6 mm²/year, about 2.7 mm²/year, about 2.8 mm²/year, about 2.9 mm²/year, about 3.0 mm²/year, about 3.5 mm²/year, about 4 mm²/year, about 4.5 mm²/year, about 5 mm²/year, about 5.5 mm²/year, about 6 mm²/year, about 6.5 mm²/year, about 7 mm²/year, about 7.5 mm²/year, or about 8.0 mm²/year) from baseline as measured FAF in (a) the at least one eye; (b) the fellow eye if GA is present at the fellow eye at baseline; or (c) both the at least one eye and the fellow eye, regardless of baseline GA status in the at least one eye and the fellow eye.

In some embodiments, the slowing of the progression of the GA further includes a reduction in (3) a mean increase (by about 0.01 mm², about 0.05 mm², about 0.1 mm², about 0.15 mm², about 0.2 mm², about 0.3 mm², about 0.4 mm², about 0.5 mm², about 0.6 mm² about 0.7 mm², about 0.8 mm², about 0.9 mm², about 1 mm², about 1.1 mm², about 1.2 mm², about 1.3 mm², about 1.4 mm², about 1.5 mm², about 1.6 mm², about 1.7 mm², about 1.8 mm², about 1.9 mm², about 2 mm², about 2.1 mm², about 2.2 mm², about 2.3 mm², about 2.4 mm², about 2.5 mm², about 2.6 mm², about 2.7 mm², about 2.8 mm², about 2.9 mm², about 3.0 mm², about 3.5 mm², about 4 mm², about 4.5 mm², about 5 mm², about 5.5 mm², about 6 mm², about 6.5 mm², about 7 mm², about 7.5 mm², or about 8.0 mm²) and a mean percent increase (by about 10%, by about 20%, by about 30%, by about 40%, by about 50%, by about 60%, by about 70%, by about 80%, or by about 90%, or by 100% or more) in a total GA lesion area from baseline as measured by FAF in (a) the at least one eye; (b) a fellow eye if GA is present at the fellow eye at baseline; or (c) both the at least one eye and the fellow eye, regardless of baseline GA status; and the reversing the progression of the GA further includes a mean decrease (by about 0.01 mm², about 0.05 mm², about 0.1 mm², about 0.15 mm², about 0.2 mm², about 0.3 mm², about 0.4 mm², about 0.5 mm², about 0.6 mm². about 0.7 mm², about 0.8 mm², about 0.9 mm², about 1 mm², about 1.1 mm², about 1.2 mm², about 1.3 mm², about 1.4 mm², about 1.5 mm², about 1.6 mm², about 1.7 mm², about 1.8 mm², about 1.9 mm², about 2 mm², about 2.1 mm², about 2.2 mm², about 2.3 mm², about 2.4 mm², about 2.5 mm², about 2.6 mm², about 2.7 mm², about 2.8 mm², about 2.9 mm², about 3.0 mm², about 3.5 mm², about 4 mm², about 4.5 mm², about 5 mm², about 5.5 mm², about 6 mm², about 6.5 mm², about 7 mm², about 7.5 mm², or about 8.0 mm²) and/or a mean percentage decrease (by about 10%, by about 20%, by about 30%, by about 40%, by about 50%, by about 60%, by about 70%, by about 80%, by about 90%, or by 100%) in the total GA lesion area from baseline as measured FAF in (a) the at least one eye; (b) the fellow eye if GA is present at the fellow eye at baseline; or (c) both the at least one eye and the fellow eye, regardless of baseline GA status in the at least one eye and the fellow eye.

In some embodiments, the slowing of the progression of the GA further includes (4) a reduction in a mean increase (by about 0.1 mm, about 0.2 mm, about 0.3 mm, about 0.4 mm, about 0.5 mm, about 0.6 mm, about 0.7 mm, about 0.8 mm, about 0.9 mm, about 1 mm, about 1.1 mm, about 1.2 mm, about 1.3 mm, about 1.4 mm, about 1.5 mm, about 1.6 mm, about 1.7 mm, about 1.8 mm, about 1.9 mm, about 2 mm, about 2.1 mm, about 2.2 mm, about 2.3 mm, about 2.4 mm, about 2.5 mm, about 2.6 mm, about 2.7 mm, or about 2.8 mm) and/or a mean percent increase (by about 10%, by about 20%, by about 30%, by about 40%, by about 50%, by about 60%, by about 70%, by about 80%, or by about 90%, or by 100% or more) in a square root of a total GA lesion area from baseline as measured by FAF in (a) the at least one eye; (b) a fellow eye if GA is present at the fellow eye at baseline; or (c) both the at least one eye and the fellow eye, regardless of baseline GA status; and the reversing the progression of the GA further includes a mean decrease (e.g., by about 0.1 mm, about 0.2 mm, about 0.3 mm, about 0.4 mm, about 0.5 mm, about 0.6 mm, about 0.7 mm, about 0.8 mm, about 0.9 mm, about 1 mm, about 1.1 mm, about 1.2 mm, about 1.3 mm, about 1.4 mm, about 1.5 mm, about 1.6 mm, about 1.7 mm, about 1.8 mm, about 1.9 mm, about 2 mm, about 2.1 mm, about 2.2 mm, about 2.3 mm, about 2.4 mm, about 2.5 mm, about 2.6 m, about 2.7 mm, or about 2.8 mm) and/or a mean percentage decrease (by about 10%, by about 20%, by about 30%, by about 40%, by about 50%, by about 60%, by about 70%, by about 80%, by about 90%, or by 100%) in the square root of the total GA lesion area from baseline as measured FAF in (a) the at least one eye; (b) the fellow eye if GA is present at the fellow eye at baseline; or (c) both the at least one eye and the fellow eye, regardless of baseline GA status in the at least one eye and the fellow eye.

In some embodiments, the treatment of GA secondary to AMD further includes (5) an improvement in one or more of (a) a monocular best-corrected visual acuity (BCVA) score in the at least one eye and/or a fellow eye from baseline as assessed by Early Treatment Diabetic Retinopathy Study (ETDRS) chart; (b) a monocular low luminance visual acuity (LLVA) score in the at least one eye and/or a fellow eye from baseline as assessed by ETDRS chart; (c) a low luminance deficit (LLD) score in the at least one eye and/or fellow eye from baseline, e.g., as assessed by ETDRS chart; (d) a National Eye Institute Visual Function Questionnaire, 25-item versions (NEI VFQ-25) score from baseline; (e) an EQ-5D-5L score in one or more of mobility, usual activities, self-care, pain or discomfort, and anxiety/depression; (f) a Lawson Instrumental Activities of Daily Living (IADL) score from baseline; (g) monocular reading speed as assessed by a Minnesota Low-Vision Reading Test (MNRead) or with Radner Reading Cards, e.g., in the at least one eye or a fellow eye; and (h) binocular reading speed as assessed by MNRead or with Radner Reading Cards. The improvement may be measured in a treated subject or in a population of treated subjects, and may be determined based on a comparison with a control, e.g., untreated or placebo-treated subjects. The mean improvement in (a), (b), and/or (c) may also be determined based on, e.g., a comparison between the at least one eye and a fellow eye, which may or may not be GA-presenting.

In some embodiments, the slowing of the progression of the GA further includes (6) a reduction in a mean rate of increase in ellipsoid zone loss area within and outside GA in a predetermined area centered on the fovea from baseline as measured by spectral-domain optical coherence tomography (SD-OCT) in (a) the at least one eye, (b) a fellow eye if GA is present at the fellow eye at baseline, or (c) both eyes; and the reversing the progression of the GA further includes a mean decrease (e.g., an increase in a mean rate of decrease) in ellipsoid zone loss area within and outside GA in a predetermined area centered on the fovea (e.g., an increase in a mean rate of decrease in ellipsoid zone loss area within and outside GA in a predetermined area centered on the fovea) from baseline as measured by SD-OCT.

In some embodiments, the slowing or reversing the progressing of the GA includes a change, e.g., a reduction or an improvement, in at least 2, 3, 4, 5, or all 6 of the above parameters.

In some embodiments, in which the subject has incomplete retinal pigment epithelium and outer retinal atrophy (iRORA) in the at least one eye and/or a fellow eye, and the treatment of the GA secondary to AMD further includes reducing the risk of the iRORA in the fellow eye converting into complete retinal pigment epithelium and outer retinal atrophy (cRORA) as determined by SD-OCT.

In some embodiments, in which the subject has a high-risk drusen in a fellow eye, and the treatment of the GA secondary to AMD further includes reducing the risk of the high-risk drusen in the fellow eye converting into late AMD as determined by SD-OCT.

In some embodiments, in which the subject has intermediate AMD (iAMD) in the at least one eye and/or a fellow eye, and the treatment of the GA secondary to AMD further includes reducing the risk of the iAMD converting into late AMD as determined by SD-OCT.

In some embodiments, in which the subject does not have GA in a fellow eye, and the treatment of GA secondary to AMD further includes a reduction in a mean increase in drusen, e.g., drusden surface area (e.g., by about 0.05 mm², about 0.1 mm², about 0.15 mm², about 0.2 mm², about 0.25 mm², about 0.3 mm², about 0.35 mm², about 0.4 mm², about 0.45 mm², about 0.5 mm², about 0.55 mm², about 0.6 mm², or about 0.65 mm²) and volume (e.g., by about 0.01 mm³, about 0.02 mm³, about 0.05 mm³, about 0.1 mm³, about 0.15 mm³, about 0.2 mm³, about 0.25 mm³, or about 0.3 mm³) in (a) the fellow eye and/or (b) both eyes combined, e.g., compared to baseline.

In some embodiments, the slowing of the progression of the GA further includes a reduction in a mean increase in a total GA lesion area as measured by SD-OCT in (a) the at least one eye; (b) a fellow eye if GA is present at the fellow eye at baseline; or (c) both eyes combined, regardless of baseline GA status of the at least one eye and the fellow eye; and the reversing of the progression of the GA further includes a mean decrease in total GA lesion area as measured by SD-OCT in (a) the at least one eye; (b) a fellow eye if GA is present at the fellow eye at baseline; or (c) both eyes combined, regardless of baseline GA status in the at least one eye and the fellow eye.

In some embodiments, the GA secondary to AMD includes extrafoveal GA, and the subject can detect a fixation target. The subject may require ≤30 minutes to complete a microperimetry test for each eye, and where the reliability test ratio is ≤20%. In some embodiments, in which the GA secondary to AMD includes extrafoveal GA, the slowing of the progression of the GA secondary to AMD further includes a reduction in a decrease in macular sensitivity from baseline as assessed by mesopic microperimetry, and the reversing of the progression of the GA further comprises an increase in macular sensitivity from baseline as assessed by mesopic microperimetry. In some embodiments, in which the GA secondary AMD includes extrafoveal GA, the slowing of the progression of the GA further includes a reduction in an increase in a number of scotomatous points from baseline as assessed by mesopic microperimetry, and the reversing the progression of the GA further includes a decrease in the number of scotomatous points from baseline as assessed by mesopic microperimetry.

In some embodiments, the GA comprises a GA lesion >1 μm outside of the foveal center outside of a foveal center.

In some embodiments, the slowing or reversing of the progression of the GA is determined based on a comparison between the subject and a control, e.g., untreated or placebo-treated subjects.

In some embodiments, the slowing or reversing of the progression of the GA or the reducing the GA is determined based on a comparison between the at least one eye and a fellow eye.

In some embodiments, the eye with GA secondary to AMD has not received an intravitreal anti vascular endothelial growth factor (VEGF) injection for intraocular vascular disease prior to receiving treatment.

In some embodiments, the eye with GA secondary to AMD has not received an intravitreal anti VEGF injection prior to receiving treatment.

In a second aspect, the present disclosure provides a method of treatment, the method including treating intermediate AMD (iAMD) in at least one eye in a subject, said treating including administering to the subject an effective amount of Compound 1 or a pharmaceutically acceptable salt thereof, wherein the treatment comprises reducing the risk of the iAMD converting into late AMD as measured by SD-OCT. In some embodiments, the subject has iAMD in both eyes.

In some embodiments, the iAMD includes IRORA, and the late AMD includes cRORA. In some embodiments, the iAMD includes a high-risk drusen.

In some embodiments, Compound 1 or the pharmaceutically acceptable salt thereof is administered in a dosing regimen of about 50-250 mg BID (e.g., about 100 mg BID or about 200 mg BID).

In some embodiments, Compound 1 or the pharmaceutically acceptable salt thereof is administered in a dosing regimen of about 200 mg to about 800 mg QD (e.g., about 400 mg QD).

In some embodiments, Compound 1 or the pharmaceutically acceptable salt thereof is systemically administered (e.g., orally or subcutaneously administered). In some embodiments, Compound 1 or the pharmaceutically acceptable salt thereof is ophthalmically administered.

In some embodiments, the subject is at least about 60 years of age (e.g., at least about 65 years of age, at least about 70 years of age, at least about 75 years of age, or at least about 80 years of age).

In some embodiments, the iAMD does not convert into late AMD within a 104-week treatment period (e.g., a 78-week treatment period, a 52-week treatment period, or a 26-week treatment period).

In some embodiments of the foregoing aspects, the subject has a glomerular filtration rate ≥30 mL/min/1.73 m².

In some embodiments of the foregoing aspects, the subject has been vaccinated against meningococcal infections within 3 years prior to receiving treatment, or has been vaccinated against meningococcal infection within less than 2 weeks prior to receiving treatment, and is further administered a prophylactic antibiotic until two weeks after vaccination.

In some embodiments of the foregoing aspects, the at least one eye has a visual acuity (VA) score of 84 to 4 letters or 20/20 to 20/800 (e.g., 84 to 24 letters or 20/20 to 20/320) as determined using ETDRS charts at starting distance of 4 meters.

In some embodiments of the foregoing aspects, the subject has not received any complement, stem cell, or gene therapy for any ophthalmological condition prior to receiving treatment.

In some embodiments of the foregoing aspects, the subject has not received any stem cell or gene therapy for any ophthalmological condition prior to receiving treatment.

In some embodiments of the foregoing aspects, the subject has not received treatment for drusen, nascent geographic atrophy (GA), or GA via any route of administration in either eye.

In some embodiments of the foregoing aspects, the subject has not received laser photocoagulation therapy for nAMD, diametric macular edema, retinal vein occlusion, and/or proliferative diabetic retinopathy in either eye.

In some embodiments, the subject has not received laser photocoagulation therapy for nAMD, diametric macular edema, retinal vein occlusion, and/or proliferative diabetic retinopathy in the study eye.

In some embodiments, the subject has not received photodynamic therapy (e.g., visudyne) or transpupillary thermotherapy in the study eye.

In some embodiments, the subject has not received external bean radiation therapy and/or any other irradiation (e.g., isotope, charged particle, photon, or x-ray) to the study and respective orbit, head, and/or neck,

In some embodiments of the foregoing aspects, the subject has not received photodynamic therapy (e.g., visudyne) in either eye; internal beam radiation therapy and/or any other irradiation (e.g., isotope, charged particle, photon, or x-ray) to the eye, orbit, head, and or neck; or transpupillary thermotherapy in either eye.

In some embodiments of the foregoing aspects, the subject has not received intravitreal delivery of steroid, anti-complement, or device implantation in either eye, provided that the intravitreal steroid delivery is not for cystoid macular edema after cataract ≥3 months.

In some embodiments, the subject has not received intravitreal delivery of steroid or device implantation in either eye, provided that the intravitreal steroid delivery is not for cystoid macular edema after cataract ≥3 months.

In some embodiments of the foregoing aspects, the subject does not have a history of recurrent infectious or inflammatory eye disease in either eye.

In some embodiments of the foregoing aspects, the subject does not have a history of retinal detachment or macular hole in either eye.

In some embodiments of the foregoing aspects, the subject does not have a history of glaucoma-filtering surgery in either eye.

In some embodiments of the foregoing aspects, the subject does not have a history of corneal transplantation in either eye.

In some embodiments of the foregoing aspects, the subject does not have a history of vitrectomy, submacular surgery, or any surgical intervention for AMD in either eye.

In some embodiments of the foregoing aspects, the subject has not received intraocular surgery within 3 months prior to treatment.

In some embodiments of the foregoing aspects, the subject does not have a known or suspected complement deficiency.

In some embodiments of the foregoing aspects, the subject does not have a history of N meningitidis infection.

In some embodiments of the foregoing aspects, the subject does not have or exhibit signs of an active bacterial, viral, or other infection; does not have a body temperature of >38° C. on two consecutive days, and does not have any febrile illness within 14 days prior to receiving treatment.

In some embodiments of the foregoing aspects, the subject does not have a history of a malignant disease within 5 years prior to treatment or an ongoing malignant disease, provided that the malignant disease is not a basal cell or squamous cell carcinoma of the skin that has been completely excised and/or cured.

In some embodiments of the foregoing aspects, the subject does not have an ALT, aspartate aminotransferase (AST), alkaline phosphatase (ALP), or direct bilirubin >2× upper limit of normal (ULN); or has a direct bilirubin level >2× ULN, provided that the subject has Gilbert's Syndrome.

In some embodiments of the foregoing aspects, the subject is not exhibiting signs of hepatobiliary cholestasis.

In some embodiments of the foregoing aspects, the subject is not exhibiting signs of a hepatitis B viral infection with negative surface antibodies.

In some embodiments of the foregoing aspects, the subject is not exhibiting signs of a hepatitis C viral infection.

In some embodiments of the foregoing aspects, the subject is not exhibiting signs of a human immunodeficiency viral infection.

In a third aspect, the present disclosure provides a use of Compound 1 or a pharmaceutically acceptable salt thereof in the manufacture of a medicament for use in any of the methods disclosed herein, e.g., treating GA secondary to AMD and/or iAMD.

In a fourth aspect, the present disclosure provides Compound 1 or a pharmaceutically acceptable salt thereof for use in any of the methods disclosed herein, e.g., treating GA secondary to AMD and/or iAMD.

In a fifth aspect, the present disclosure provides a kit for treating GA secondary to AMD and/or iAMD in at least one eye in a subject. The kit includes (a) a dose of Compound 1 or a pharmaceutically acceptable salt thereof; and (b) instructions for using Compound 1 or the pharmaceutically acceptable salt thereof according to the any of the methods disclosed herein.

### BRIEF DESCRIPTION OF THE DRAWING

FIG. 1 is a schematic depicting the design of a Phase II clinical trial described in Example 1 (bid: twice daily; qd: once daily; IA: interim analysis; EOS: end of study). The futility analysis (interim analysis 1 [IA1]) may be conducted when approximately 50% patients complete Week 28 visit. The futility and dose-response analyses (interim analysis 2 [IA2] may be conducted when approximately 50% patients complete Week 52 visit. If the dose-response is positive, pairwise comparisons may be performed at IA2. Placebo patients are transitioned after 52 weeks to the optimal dose, or, if not identified, are re-randomized at Week 52 to 1 of the active treatment groups until the optimal dose has been determined.

### DETAILED DESCRIPTION

### Definitions

As used herein, the word “a” or “plurality” before a noun represents one or more of the particular nouns. For example, the phrase “a mammalian cell” represents “one or more mammalian cells.” The singular form “a,” “an,” and “the” include plural reference unless the context clearly dictates otherwise.

The term “about”, particularly in reference to a given quantity or number, is meant to encompass deviations within plus or minus ten percent (+10%). (e.g., +5%).

As used herein, the term “baseline” refers to a parameter (e.g., a level, a score, or an anatomical measure) detected or measured in a subject at the start of a treatment regimen.

As used herein, “effective treatment” refers to treatment producing a beneficial effect, e.g., the slowing or reversing of an unwanted condition (e.g., GA secondary to AMD). A beneficial effect can take the form of an improvement of one or more clinical symptoms or parameters (e.g., GA lesion area) associated with the unwanted condition over baseline, i.e., an improvement over a measurement or observation made prior to initiation of therapy.

The term “effective amount” or “therapeutically effective amount” refers to an amount of an agent that provides the desired biological, therapeutic, and/or prophylactic result. That result can be reduction, amelioration, palliation, lessening, delaying and/or alleviation of one or more of the signs, symptoms, or causes of a disease, or any other desired alteration of a biological system. In one example, an “effective amount” is the amount of Compound 1 or a pharmaceutically acceptable salt thereof useful, e.g., clinically proven, to slow (e.g., halt) the progression of an unwanted condition (e.g., GA secondary to AMD) or reverse the progression of the unwanted condition (e.g., reversing the GA secondary to AMD). An effective amount can be provided in one or more administrations.

As is customary in the field, the clinical parameters used herein may be expressed in any conventional manner. For instance, changes (e.g., increase or decrease) in the GA lesion area may be expressed absolute terms (e.g., mm²), derivative term (e.g., square root (sqrt) in mm), or a relative term (e.g., % change or fold change). Moreover, changes to the clinical parameter may be determined based on subject specific characteristics and/or population characteristics. For example, mean (or median) GA lesion area may be derived from multiple measurements in a treated subject(s) (e.g., at least 3, at least 5, at least 10, at least 20, or more, e.g., at least 40 different, preferably predetermined, sites in a retinal scan) or from measurements in a population of treated subjects (e.g., at least 3, at least 5, at least 10, at least 20, at least 30, at least 40, at least 50, at least 60, at least 70, or at least 80, or more, e.g., 300 subjects, in which multiple measurements may be made in each treated subject as described above).

As used herein, the term “improvement,” in reference to a score (e.g., in a visual acuity test, a reading speed test, an NEI VFQ-25 questionnaire, an EQ-5D-5L questionnaire, or a Lawton IADL scale), refers to an improvement in the score observed in a subject(s) treated according to a method disclosed herein as compared to a control, where the improvement may be observed in a particular treated subject or a population of treated subjects (e.g., at least 3, at least 5, at least 10, at least 20, at least 30, at least 40, at least 50, at least 60, at least 70, or at least 80, or more, e.g., 300 treated subjects). In some embodiments, the control is untreated subjects (e.g., at least 3, at least 5, at least 10, at least 20, at least 30, at least 40, at least 50, at least 60, at least 70, or at least 80, or more, e.g., 300 untreated subjects). In some embodiments, the control is placebo-treated subjects (e.g., at least 3, at least 5, at least 10, at least 20, at least 30, at least 40, at least 50, at least 60, at least 70, or at least 80, or more, e.g., 300 placebo-treated subjects).

As used herein, the term “mean percent increase/decrease in a square root of a total GA lesion area” refers to a percent increase/decrease in the square root of the total GA lesion area (e.g., decrease by at least 10%, at least 20%, at least 30%, at least 40%, at least 50%, at least 60%, at least 70%, at least 80%, at least 90%, or at least 99%; or increase by less than 500%, less than 400%, less than 300%, less than 250%, less than 200%, less than 150%, less than 120%, less than 100%, less than 90%, less than 80%, less than 70%, less than 60%, less than 50%, less than 40%, less than 30%, less than 20%, or less than 10%) in a GA-presenting eye(s) in a subject(s) treated according to a method disclosed herein as compared to a control, as measured by any method known in the art (e.g., FAF and/or SD-OCT), derived from multiple measurements in the treated subject(s) (e.g., at least 3, at least 5, at least 10, at least 20, or more, e.g., at least 40 different, preferably predetermined, sites in a retinal scan) or from measurements in a population of treated subjects (e.g., at least 3, at least 5, at least 10, at least 20, at least 30, at least 40, at least 50, at least 60, at least 70, or at least 80, or more, e.g., 300 subjects, in which multiple measurements may be made in each treated subject as described above). In some embodiments, the control is a fellow eye of the treated subject (which may or may not be GA-presenting at baseline). In some embodiments, the control is untreated subjects. In some embodiments, the control is placebo-treated subjects. In some embodiments, the mean percent increase/decrease is derived from multiple measurements in a treated subject.

As used herein, the term “mean percent increase/decrease in a total GA lesion area” refers to a percent increase/decrease in the total GA lesion area (e.g., decrease by at least 10%, at least 20%, at least 30%, at least 40%, at least 50%, at least 60%, at least 70%, at least 80%, at least 90%, or at least 99%; or increase by less than 500%, less than 400%, less than 300%, less than 250%, less than 200%, less than 150%, less than 120%, less than 100%, less than 90%, less than 80%, less than 70%, less than 60%, less than 50%, less than 40%, less than 30%, less than 20%, or less than 10%) in a subject(s) treated according to a method disclosed herein as compared to a control, as measured by any method known in the art (e.g., FAF and/or SD-OCT), derived from multiple measurements in the treated subject(s) (e.g., at least 3, at least 5, at least 10, at least 20, or more, e.g., at least 40 different, preferably predetermined, sites in a retinal scan) or from measurements in a population of treated subjects (e.g., at least 3, at least 5, at least 10, at least 20, at least 30, at least 40, at least 50, at least 60, at least 70, or at least 80, or more, e.g., 300 subjects, in which multiple measurements may be made in each treated subject as described above). In some embodiments, the control is a fellow eye of the treated subject (which may or may not be GA-presenting at baseline). In some embodiments, the control is untreated subjects (e.g., at least 3, at least 5, at least 10, at least 20, at least 30, at least 40, at least 50, at least 60, at least 70, or at least 80, or more, e.g., 300 untreated subjects, in which multiple measurements may be made in each untreated subject). In some embodiments, the control is placebo-treated subjects (e.g., at least 3, at least 5, at least 10, at least 20, at least 30, at least 40, at least 50, at least 60, at least 70, or at least 80, or more, e.g., 300 placebo-treated subjects, in which multiple measurements may be made in each placebo-treated subject). In some embodiments, the mean percent increase/decrease is derived from multiple measurements in a treated subject.

As used herein, the term “mean increase/decrease in a square root of a total GA lesion area” refers to an increase/decrease in the square root of the total GA lesion area (e.g., over a baseline of about 2.8±0.72 mm, as measured by FAF in control subjects) in a GA-presenting eye(s) in a subject(s) treated according to a method disclosed herein as compared to a control, as measured by any method known in the art (e.g., FAF and/or SD-OCT), derived from multiple measurements in the treated subject (e.g., at least 3, at least 5, at least 10, at least 20, or more, e.g., at least 40 different, preferably predetermined, sites in a retinal scan) or from measurements in a population of treated subject(s) (e.g., at least 3, at least 5, at least 10, at least 20, at least 30, at least 40, at least 50, at least 60, at least 70, or at least 80, or more, e.g., 300 subjects, in which multiple measurements may be made in each treated subject as described above). For instance, quantitative measurement of total GA lesion area progression by FAF imaging may be used as an anatomic marker used as clinical endpoint for clinical studies (Holz et al., Am J Ophthalmol. 2007; 143(3):463-472; Holz et al., JAMA Ophthalmol. 2018; 136(6):666-677; Liao et al., Ophthalmology. 2020; 127(2):186-195; Sadda et al., Retina. 2016; 36(10):1806-1822) and particularly, square root (sqrt) transformation of the GA lesion area measurements may be used to eliminate variability dependence on baseline lesion size (Feuer et al., JAMA Ophthalmology. 2013; 131(1):110-111; Yehoshua et al., Ophthalmology. 2014; 121(3):693-701). In some embodiments, the control is a fellow eye of the treated subject (which may or may not be GA-presenting at baseline). In some embodiments, the control is untreated subject (e.g., at least 3, at least 5, at least 10, at least 20, at least 30, at least 40, at least 50, at least 60, at least 70, or at least 80, or more, e.g., 300 untreated subjects, in which multiple measurements may be made in each untreated subject). In some embodiments, the control is placebo-treated subjects (e.g., at least 3, at least 5, at least 10, at least 20, at least 30, at least 40, at least 50, at least 60, at least 70, or at least 80, or more, e.g., 300 placebo-treated subjects, in which multiple measurements may be made in each placebo-treated subject). In some embodiments, the mean increase/decrease is derived from multiple measurements in a treated subject

As used herein, the term “mean rate of increase/decrease in a square root of a total GA lesion area” refers to a rate of increase/decrease in the square root of the total GA lesion area (e.g., over a baseline of about 0.35±0.05 mm/year, as measured by FAF in control subjects) in a subject(s) treated according to a method disclosed herein as compared to a control, as measured by any method known in the art (e.g., FAF and/or SD-OCT), derived from multiple measurements in the treated subject(s) (e.g., at least 3, at least 5, at least 10, at least 20, or more, e.g., at least 40 different, preferably predetermined, sites in a retinal scan) or from measurements in a population of treated subjects (e.g., at least 3, at least 5, at least 10, at least 20, at least 30, at least 40, at least 50, at least 60, at least 70, or at least 80, or more, e.g., 300 subjects, in which multiple measurements may be made in each treated subject as described above). See, Holz (2007); Holz (2018); Liao (2020): Sadda (2016), supra. In some embodiments, the control is a fellow eye of the treated subject (which may or may not be GA-presenting at baseline). In some embodiments, the control is untreated subjects (e.g., at least 3, at least 5, at least 10, at least 20, at least 30, at least 40, at least 50, at least 60, at least 70, or at least 80, or more, e.g., 300 untreated subjects, in which multiple measurements may be made in each untreated subject). In some embodiments, the control is placebo-treated subjects (e.g., at least 3, at least 5, at least 10, at least 20, at least 30, at least 40, at least 50, at least 60, at least 70, or at least 80, or more, e.g., 300 placebo-treated subjects, in which multiple measurements may be made in each placebo-treated subject). In some embodiments, the mean rate of increase/decrease is derived from multiple measurements in a treated subject

As used herein, the term “mean increase/decrease in a total GA lesion area” refers to an increase/decrease in total GA lesion area (e.g., over a baseline of about 8.2±4.05 mm², as measured by FAF in control subjects) in a subject(s) treated according to a method disclosed herein as compared to a control, as measured by any method known in the art (e.g., FAF and/or SD-OCT), derived from multiple measurements in the treated subject(s) (e.g., at least 3, at least 5, at least 10, at least 20, or more, e.g., at least 40 different, preferably predetermined, sites in a retinal scan) or from measurements in a population of treated subjects (e.g., at least 3, at least 5, at least 10, at least 20, at least 30, at least 40, at least 50, at least 60, at least 70, or at least 80, or more, e.g., 300 treated subjects, in which multiple measurements may be made in each treated subject as described above). See, Holz (2007); Holz (2018); Liao (2020); Sadda (2016), supra. In some embodiments, the control is a fellow eye of the treated subject (which may or may not be GA-presenting at baseline). In some embodiments, the control is untreated subjects (e.g., at least 3, at least 5, at least 10, at least 20, at least 30, at least 40, at least 50, at least 60, at least 70, or at least 80, or more, e.g., 300 untreated subjects, in which multiple measurements may be made in each untreated subject). In some embodiments, the control is placebo-treated subjects (e.g., at least 3, at least 5, at least 10, at least 20, at least 30, at least 40, at least 50, at least 60, at least 70, or at least 80, or more, e.g., 300 placebo-treated subjects, in which multiple measurements may be made in each placebo-treated subject). In some embodiments, the mean increase/decrease is derived from multiple measurements in a treated subject

As used herein, the term “mean rate of increase/decrease in a total GA lesion area” refers to a rate of increase/decrease in total GA lesion area in a subject(s) treated according to a method disclosed herein as compared to a control, as measured by any method known in the art (e.g., FAF and/or SD-OCT), derived from multiple measurements in the treated subject(s) (e.g., at least 3, at least 5, at least 10, at least 20, or more, e.g., at least 40 different, preferably predetermined, sites in a retinal scan) or from measurements in a population of treated subjects (e.g., at least 3, at least 5, at least 10, at least 20, at least 30, at least 40, at least 50, at least 60, at least 70, or at least 80, or more, e.g., 300 treated subjects, in which multiple measurements may be made in each treated subject as described above). See, Holz (2007); Holz (2018); Liao (2020); Sadda (2016), supra. For example, mean rate of increase in total GA lesion area can be measured using a linear regression model of a curve that plots total GA area (y-axis) and time in years (x-axis). In some embodiments, the control is a fellow eye of the treated subject (which may or may not be GA-presenting at baseline). In some embodiments, the control is untreated subjects (e.g., at least 3, at least 5, at least 10, at least 20, at least 30, at least 40, at least 50, at least 60, at least 70, or at least 80, or more, e.g., 300 untreated subjects, in which multiple measurements may be made in each untreated subject). In some embodiments, the control is placebo-treated subjects (e.g., at least 3, at least 5, at least 10, at least 20, at least 30, at least 40, at least 50, at least 60, at least 70, or at least 80, or more, e.g., 300 placebo-treated subjects, in which multiple measurements may be made in each placebo-treated subject). In some embodiments, the mean increase/decrease is derived from multiple measurements in a treated subject

As used herein, the term “mean increase/decrease in drusen surface area and/or volume” refers to an average in the increase/decrease in drusen surface area and/or volume (e.g., change relative to a cutoff level of about 0.03 mm³) in a subject(s) treated according to a method disclosed herein as compared to a control, as measured by any method known in the art (e.g., FAF and/or SD-OCT). For instance, drusen surface area may be measured using FAF and SD-OCT images, and drusen volume is derived using SD-OCT images by masked graders per the Central Reading Manual at specific time points. See Abdelfattah et al., Invest Ophthalmol Vis Sci. 2016; 57(4):1839-1846; Folgar et al., Ophthalmology. 2016; 123(1):39-50.e31. In some embodiments, the control is a fellow eye of the treated subjects. In some embodiments, the control is untreated subjects. In some embodiments, the control is placebo-treated subjects.

As used herein, the term “mean rate of increase/decrease in ellipsoid zone loss area within and outside GA in a predetermined area centered on the fovea” refers to a rate of increase/decrease in ellipsoid zone loss area within and outside GA in a predetermined area centered on the fovea in a subject(s) treated according to a method disclosed herein as compared to a control, as measured by any method known in the art (e.g., FAF and/or SD-OCT), derived from multiple measurements in the treated subject (e.g., at least 3, at least 5, at least 10, at least 20, or more, e.g., at least 40 different, preferably predetermined, sites in a retinal scan) or from measurements in a population of treated subjects (e.g., at least 3, at least 5, at least 10, at least 20, at least 30, at least 40, at least 50, at least 60, at least 70, or at least 80, or more, e.g., 300 treated subjects, in which multiple measurements may be made in each treated subject as described above). In some embodiments, a quantitative optical coherence tomographic (OCT) approach may be used to determine the relative intensity of the ellipsoid zone (EZ), by expressing a ratio of the EZ to the external limit membrane (ELM). See, Pfau et al., Retina. 2020; 40(1):169-180. Other methods, e.g., as described in Lara-Medina et al., IntechOpen; 2019; Mukherjee et al., Invest Ophthalmol Vis Sci. 2017; 58(6): Bio291; and Tao et al., Clin Exp Ophthalmol. 2016; 44(5):422-430, may also be used. In some embodiments, the control is a fellow eye of the treated subject (which may or may not be GA-presenting at baseline). In some embodiments, the control is untreated subjects (e.g., at least 3, at least 5, at least 10, at least 20, at least 30, at least 40, at least 50, at least 60, at least 70, or at least 80, or more, e.g., 300 untreated subjects, in which multiple measurements may be made in each untreated subject). In some embodiments, the control is placebo-treated subjects (e.g., at least 3, at least 5, at least 10, at least 20, at least 30, at least 40, at least 50, at least 60, at least 70, or at least 80, or more, e.g., 300 placebo-treated subjects, in which multiple measurements may be made in each placebo-treated subject). In some embodiments, the mean rate of increase/decrease is derived from multiple measurements in a treated subject.

As used herein, the term “mean increase/decrease in ellipsoid zone loss area within and outside GA in a predetermined area centered on the fovea” refers to an increase/decrease in ellipsoid zone loss area within and outside GA in a predetermined area centered on the fovea in a subject(s) treated according to a method disclosed herein as compared to a control, as measured by any method known in the art (e.g., FAF and/or SD-OCT), derived from multiple measurements in the treated subject(s) (e.g., at least 3, at least 5, at least 10, at least 20, or more, e.g., at least 40 different, preferably predetermined, sites in a retinal scan) or from measurements in a population of treated subjects (e.g., at least 3, at least 5, at least 10, at least 20, at least 30, at least 40, at least 50, at least 60, at least 70, or at least 80, or more, e.g., 300 treated subjects, in which multiple measurements may be made in each treated subject as described above). Representative methods are described in Mukherjee et al. (2017); Pfau et al. (2020); and Tao et al. (2016), supra. In some embodiments, the control is a fellow eye of the treated subject (which may or may not be GA-presenting at baseline). In some embodiments, the control is untreated subjects (e.g., at least 3, at least 5, at least 10, at least 20, at least 30, at least 40, at least 50, at least 60, at least 70, or at least 80, or more, e.g., 300 untreated subjects, in which multiple measurements may be made in each untreated subject). In some embodiments, the control is placebo-treated subjects (e.g., at least 3, at least 5, at least 10, at least 20, at least 30, at least 40, at least 50, at least 60, at least 70, or at least 80, or more, e.g., 300 placebo-treated subjects, in which multiple measurements may be made in each placebo-treated subject). In some embodiments, the mean increase/decrease is derived from multiple measurements in a treated subject.

As used herein, the term “increase/decrease in macular sensitivity” refers to an increase/decrease in macular sensitivity (e.g., as assessed by a decrease/increase in scotomatous points) in a subject (or a population of subjects, e.g., at least 3, at least 5, at least 10, at least 20, at least 30, at least 40, at least 50, at least 60, at least 70, or at least 80, or more, e.g., 300 treated subjects) treated according to a method disclosed herein as compared to a control, as measured by any method known in the art (e.g., mesopic microperimetry). A representative microperimetry technique is described in Csaky et al., Surv Ophthalmol. 2019; 64(3):353-364, wherein light stimuli of various intensities is altered to determine the lowest level at which the subject can detect the light; stimulus intensity is measured in decibels (dB), wherein a higher score indicates detection of a dimmer stimulus and thus higher retinal sensitivity; a score of 0 dB signifies an absolute scotoma (assuming there is no floor effect), reflecting a failure to detect the brightest stimulus available on the instrument. Next, eye-tracking technology is used to lock onto specific fundus features and the stimulus display is adjusted many times per second (depending on eye-tracker frequency) to compensate for eye movements and to localize stimuli to specific retinal locations, after which the scores are obtained. In some embodiments, the control is a fellow eye of the treated subject (which may or may not be GA-presenting at baseline). In some embodiments, the control is untreated subjects (e.g., at least 3, at least 5, at least 10, at least 20, at least 30, at least 40, at least 50, at least 60, at least 70, or at least 80, or more, e.g., 300 untreated subjects, in which multiple measurements may be made in each untreated subject). In some embodiments, the control is placebo-treated subjects (e.g., at least 3, at least 5, at least 10, at least 20, at least 30, at least 40, at least 50, at least 60, at least 70, or at least 80, or more, e.g., 300 placebo-treated subjects, in which multiple measurements may be made in each placebo-treated subject). In some embodiments, the mean increase/decrease is derived from multiple measurements in a treated subject.

As used herein, the term “increase/decrease in scotomatous points” refers to an average in the increase/decrease in the number of scotomatous points in a subject (or population of subjects, e.g., at least 3, at least 5, at least 10, at least 20, at least 30, at least 40, at least 50, at least 60, at least 70, or at least 80, or more, e.g., 300 treated subjects) treated according to a method disclosed herein as compared to a control, as measured by any method known in the art (e.g., mesopic microperimetry). Representative methods are described in Csaky et al. (2019), supra. In some embodiments, an increase in about 4.4 scotomatous points per year, as calculated from longitudinal data collected at 68 test points evenly distributed from the central 20° of the macula, may be observed in the GA subjects and a reduction (e.g., at least 0.5 points, at least 1 point, at least 1.5 points, at least 2 points, or more, e.g., at least 4 points), following administration of the compounds of the present disclosure, is indicative of effective treatment. In some embodiments, the control is a fellow eye of the treated subject (which may or may not be GA-presenting at baseline). In some embodiments, the control is untreated subjects (e.g., at least 3, at least 5, at least 10, at least 20, at least 30, at least 40, at least 50, at least 60, at least 70, or at least 80, or more, e.g., 300 untreated subjects, in which multiple measurements may be made in each untreated subject). In some embodiments, the control is placebo-treated subjects (e.g., at least 3, at least 5, at least 10, at least 20, at least 30, at least 40, at least 50, at least 60, at least 70, or at least 80, or more, e.g., 300 placebo-treated subjects, in which multiple measurements may be made in each placebo-treated subject).

As used herein, the term “pharmaceutically acceptable salt” represents those salts of the compounds described that are, within the scope of sound medical judgment, suitable for use in contact with the tissues of humans and animals without undue toxicity, irritation, allergic response and the like and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Handbook of Pharmaceutical Salts: Properties, Selection, and Use, (Eds. P. H. Stahl and C. G. Wermuth), Wiley-VCH, 2008. These salts may be acid addition salts involving inorganic or organic acids. The salts can be prepared in situ during the final isolation and purification of the compounds described herein or separately by reacting the free base group with a suitable acid. Methods for preparation of the appropriate salts are well-established in the art. Representative acid addition salts include acetate, adipate, alginate, ascorbate, aspartate, benzenesulfonate, benzoate, bisulfate, borate, bromide, butyrate, camphorate, camphorsulfonate, chloride, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts and the like.

As used herein, the term “pharmaceutical composition” refers to an active compound, formulated together with one or more pharmaceutically acceptable excipients. In some embodiments, a compound is present in unit dose amount appropriate for administration in a therapeutic regimen that shows a statistically significant probability of achieving a predetermined therapeutic effect when administered to a relevant population. In certain embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained release formulation; topical application, for example, as a cream, ointment, or a controlled release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

The term “pharmaceutically acceptable excipient,” as used herein, refers to any inactive ingredient (for example, a vehicle capable of suspending or dissolving the active compound) having the properties of being nontoxic and non-inflammatory in a subject. Typical excipients include, for example: antiadherents, antioxidants, binders, coatings, compression aids, disintegrants, dyes, emollients, emulsifiers, diluents, film formers or coatings, flavors, fragrances, glidants, lubricants, preservatives, printing inks, sorbents, suspending or dispersing agents, sweeteners, or waters of hydration. Excipients include, but are not limited to: butylated optionally substituted hydroxytoluene (e.g., BHT), calcium carbonate, calcium phosphate dibasic, calcium stearate, croscarmellose, crosslinked polyvinyl pyrrolidone, citric acid, crospovidone, cysteine, ethylcellulose, gelatin, optionally substituted hydroxypropyl cellulose, optionally substituted hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch, stearic acid, stearic acid, sucrose, talc, titanium dioxide, vitamin A, vitamin E, vitamin C, and xylitol. Those of ordinary skill in the art are familiar with a variety of agents and materials useful as excipients.

As used herein, the term “reducing the risk of iAMD converting into late AMD” refers to reducing the percentage of iAMD (e.g., IRORA or high-risk drusen) converting into late AMD (e.g., cRORA) in subjects treated according to any of the methods of the disclosure. The reduction is in comparison to control subjects of the same age, sex, and/or condition (e.g., comorbidities), e.g., those that are untreated or placebo-treated. In some embodiments, the percentage of iAMD converting into late AMD in subjects treated according to any of the methods of the disclosure is reduced by at least 10% (at least 20%, at least 30%, at least 40%, at least 50%, at least 60%, at least 70%, at least 80%, at least 90%, or 99% or more) relative to the percentage of iAMD converting into late AMD in control subjects.

As used herein, the term “slowing or reversing a progression of GA” in a subject refers to an observation of the slowing or reversing of the progression of GA in a subject(s) treated according to a method disclosed herein as compared to a control. In some embodiments, the control is a fellow eye of the treated subjects. In some embodiments, the control is untreated subjects (e.g., at least 10, at least 20, at least 30, at least 40, at least 50, at least 60, at least 70, or at least 80 subjects, or about the same number as the treated subjects). In some embodiments, the control is placebo-treated subjects (e.g., at least 10, at least 20, at least 30, at least 40, at least 50, at least 60, at least 70, or at least 80 subjects, or about the same number as the treated subjects).

As used herein, the term “subject” or “patient” is a human patient (e.g., a patient with GA secondary to AMD). As used herein, the terms “subject” and “patient” are interchangeable.

As used herein, the term “treating” includes therapeutic treatments. The term “therapeutic” treatment is art-recognized and includes administration to a human subject of Compound 1 or a pharmaceutically acceptable salt thereof (e.g., in a pharmaceutical composition) after manifestation of the unwanted condition (e.g., GA secondary to AMD). It is intended to diminish, ameliorate, stabilize, or slow or halt the progression of the existing unwanted condition (e.g., GA secondary to AMD) or side effects thereof. Preferably, it is intended that the progression of an unwanted condition (e.g., GA secondary to AMD) is slowed, halted, or reversed (e.g., the GA secondary to AMD is reduced) relative to a control. In the context of treating an ophthalmic disorder such as AMD (e.g., GA secondary to AMD), the control may be a fellow eye of a subject, an untreated subject, or a placebo-treated subject.

### Age-Related Macular Degeneration

The disclosure relates to methods for treating GA secondary to AMD. AMD is a complement-mediated disorder of the eye caused by a dysfunction of or excessive activation of complement factor D and impacts the central area of the retina in the eye, called the macula. AMD is a leading cause of blindness in people aged 60 and over. An estimated 17 million people worldwide are affected by AMD.

There are two main types of AMD dry (non-neovascular) form and wet (neovascular) form. Dry form AMD occurs when parts of the macula get thinner with age and yellow deposits/clumps, called drusen, grow on the macula. These clumps can get bigger and more numerous resulting in distorted vision. As the condition progresses, the light-sensitive cells in the macula get thinner and eventually die. In the atrophic form, blind spots in the center of vision occur, resulting in a loss of central vision. Wet form AMD is less common but is more serious and occurs when new abnormal blood vessels grow underneath the macula. These blood vessels may leak blood and fluid into the retina, distorting vision so that straight lines look wavy. As the condition progresses these blood vessels and their bleeding eventually form scars, leading to permanent loss of central vision.

GA is a chronic progressive degeneration of the macula, and is considered as part of late-stage AMD. The condition leads to central scotomas and permanent loss of visual acuity. The disease is characterized by localized sharply demarcated atrophy of outer retinal tissue, retinal pigment epithelium and choriocapillaris. It starts typically in the perifoveal region and expands to involve the fovea with time, leading to central scotomas and permanent loss of visual acuity. It is bilateral in most cases. Over 8 million people are affected worldwide with GA, approximately 20% of all individuals with AMD.

The clinical classification of AMD is provided in Table 1 below:

TABLE 1
Clinical Classification of AMD

| Classification | | |
|---|---|---|
| of AMD | Subcategory | Definition |
| No apparent aging | | No drusen AND |
| changes¹ | | No AMD pigmentary abnormalities² |
| Normal aging | | Only drupelets (small drusen ≤63 μm) AND |
| changes¹ | | No AMD pigmentary abnormalities² |
| Early AMD¹ | | Medium drusen >63 μm and ≤125 μm AND |
| | | No AMD pigmentary abnormalities² |
| Intermediate | | Large drusen >125 μm AND/OR |
| AMD¹ (iAMD) | | Any AMD pigmentary abnormalities² |
| | High-risk | Drusenoid lesions (drusen and drusenoid pigment |
| | drusen³ | epithelium |
| | | detachment) with heterogeneous internal reflectivity |
| | | and/or intra-retinal hyperreflective foci, with a minimum |
| | | height of 40 μm, observed in SD-OCT |
| | Non-exudative | Evidence of macular neovascularization by OCTA or |
| | neovascular | the presence of a plaque or hot spot on ICGA, but no |
| | AMD⁴ | leakage on FA and no macular fluid by structural OCT |
| | | imaging |
| | iRORA⁵ | A region of signal hypertransmission into the choroid, |
| | | AND |
| | | A corresponding zone of attenuation or disruption of |
| | | the RPE, with or without persistence of basal laminar |
| | | deposits, AND |
| | | Evidence of overlying photoreceptor degeneration, that |
| | | is, subsidence of the INL and OPL, presence of a |
| | | hyporeflective wedge in the HFL, thinning of the ONL, |
| | | disruption of the ELM, or disintegrity of the EZ, AND |
| | | when these criteria do not meet the definition of |
| | | cRORA |
| Late AMD¹ | cRORA⁶ | Region of hypertransmission of at least 250 μm in |
| (Neovascular | | diameter in any lateral dimension, AND |
| AMD AND/ | | Zone of attenuation or disruption of the RPE of at least |
| OR Any | | 250 μm in diameter, AND |
| geographic | | Evidence of overlying photoreceptor degeneration. |
| atrophy) | | Features of photoreceptor degeneration include all of |
| | | the following: |
| | | loss of the IZ, EZ, and ELM and thinning of the |
| | | ONL. |
| | | AND absence of scrolled RPE or other signs of an |
| | | RPE tear. |
| | Exudative | Evidence of macular neovascularization on OCTA and |
| | neovascular | macular fluid by structural OCT, evidence of leakage |
| | AMD⁴ | on FA, with or without evidence of a plaque or hot spot |
| | | on ICGA. |

Abbreviations: AMD = age-related macular degeneration; cRORA = complete RPE and outer retinal atrophy; ELM = external limiting membrane; HFL = Henle fiber layer ; INL = inner nuclear layer; IZ = interdigitation zone; EZ = ellipsoid zone; FA = fluorescein angiography; iAMD = intermediate AMD; ICGA = indocyanine green angiography; iRORA = incomplete RPE and outer retinal atrophy; N/A = not applicable; OCT = optical coherence tomography; OCTA = optical coherence tomography angiography; ONL = outer nuclear layer; OPL = outer plexiform layer; RPE = retinal pigment epithelium; SD-OCT = spectral-domain optical coherence tomography
¹Ferris F L, 3rd, Wilkinson C P, Bird A, et al., Ophthalmology. 2013; 120(4): 844-851
²AMD pigmentary abnormalities are any definite hyper- or hypopigmentary abnormalities associated with medium or large drusen but not associated with known disease entities
³Ouyang et al., Ophthalmology. 2013; 120(12): 2656-2665
⁴Laiginhas et al., Ophthalmol Retina. 2020; 4(7): 651-661
⁵Guymer et al., Ophthalmology. 2020; 127(3): 394-409
⁶Sadda et al., Ophtlamology. 2018; 125(4): 537-548

### Methods of Treatment

The present disclosure provides methods for treating GA secondary to AMD (e.g., slowing or reversing the progression of GA or reducing the GA) in a subject. The method includes administering to the subject a therapeutically effective of amount of Compound 1 (ALXN2040/danicopan) or a pharmaceutically acceptable thereof. In preferred embodiments, Compound 1 or the pharmaceutically acceptable salt thereof is administered orally.

In some embodiments, Compound 1 or the pharmaceutically acceptable salt thereof is administered once daily (QD). In some embodiments, Compound 1 or the pharmaceutically acceptable salt thereof is administered in a dose of about 100 mg to about 1000 mg QD (e.g., about 200 mg to about 800 mg QD, about 250 mg to about 600 mg QD, about 300 mg to about 500 mg QD, about 350 mg to about 400 mg QD, about 100 mg QD, about 150 mg QD, about 200 mg QD, about 250 mg QD, about 300 mg QD, about 350 mg QD, about 400 mg QD, about 450 mg QD, about 500 mg QD, about 550 mg QD, about 600 mg QD, about 650 mg QD, about 700 mg QD, about 750 mg QD, about 800 mg QD, about 850 mg QD, about 900 mg QD, about 950 mg QD, or about 1000 mg QD). In some embodiments, Compound 1 or the pharmaceutically acceptable salt thereof is administered twice daily (BID). In some embodiments, Compound 1 or the pharmaceutically acceptable salt thereof is administered in a dose of about 50 mg to about 500 mg QID (e.g., about 100 mg to about 400 mg BID, about 125 mg to about 300 mg BID, about 150 mg to about 250 mg BID, about 175 mg to about 200 mg BID, about 50 mg BID, about 100 mg BID, about 125 mg BID, about 150 mg BID, about 175 mg BID, about 200 mg BID, about 225 mg BID, about 250 mg BID, about 275 mg BID, about 300 mg BID, about 325 mg BID, about 350 mg BID, about 375 mg BID, about 400 mg BID, about 425 mg BID, about 450 mg BID, 475 mg BID, or about 500 mg BID).

In some embodiments, the course of treatment with Compound 1 or a pharmaceutically acceptable salt thereof lasts for 26 weeks. In some embodiments, the course of treatment lasts for 52 weeks. In some embodiments, the course of treatment lasts for 104 weeks. In some embodiments, the course of treatment lasts for 26-52, 26-78, 26-104, 26-130, 26-156, 26-182, 26-208 weeks, or more. In some embodiments, the course of treatment lasts for greater than 26, 27, 28, 29, 30, 31, 32, 33, 34, 35, 36, 37, 38, 39, 40, 41, 42, 43, 44, 45, 46, 47, 48, 49, 50, 51, 52, 78, 104, 130, 156 or 182 weeks. In some embodiments, the course of treatment lasts for greater than 1, 2, 3, 4, 5, 10, 15, 20, 25, 30, 35, 40, 45, 50, 55, 60, 65, 70, 75, 80, or more years. In some embodiments, the course of treatment lasts for the remainder of the subject's life.

In some embodiments, the first sign of effective treatment (e.g., the slowing or reversing the progression of the GA or the reducing the GA) occurs by 26 weeks of treatment with Compound 1 or a pharmaceutically acceptable salt thereof. In some embodiments, the first sign of effective treatment (e.g., the slowing or reversing of the progression of AMD, such as GA secondary to AMD) by 52 weeks of treatment with Compound 1 or a pharmaceutically acceptable salt thereof occurs. In some embodiments, the first sign of effective treatment (e.g., the slowing or reversing the progression of the GA or the reducing the GA)) by 104 weeks of treatment with Compound 1 or a pharmaceutically acceptable salt thereof occurs. In some embodiments, the first sign of effective treatment (e.g., the slowing or reversing the progression of the GA or the reducing the GA) occurs between weeks 1-26, 26-52, 52-78, 78-104, 104-130, 130-156, 156-182, or 182-208 of treatment with Compound 1 or a pharmaceutically acceptable salt thereof. In some embodiments, the first sign of effective treatment (e.g., the slowing or reversing of the progression of AMD, such as GA secondary to AMD) occurs at week 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 78, 104, 130, 156, or 182.

In some embodiments, the sign of effective treatment (e.g., the slowing or reversing of the progression of the GA or the reducing the GA) includes one or more of (1) a reduction in the rate of increase in the square root of the total GA lesion area from baseline, (2) a reduction in the rate of increase in the total GA lesion area from baseline in one or both eyes, (3) a reduction in the increase (e.g., or percent increase) in the total GA lesion area from baseline in one or both eyes or a decrease in the total GA lesion area in one or both eyes, (4) a reduction in the increase (e.g., percent increase) in the square root of the total GA lesion area from baseline in one or both eyes or a decrease in the square root of the total GA lesion area from baseline in one or both eyes, and (5) a reduction in increase in drusen surface area and/or volume in one or both eyes, as measured by FAF and/or SD-OCT, e.g., as compared to untreated or placebo-treated subjects. The sign of effective treatment may also be based on a comparison between a GA-presenting eye and a fellow eye that may or may not be GA-presenting.

In some embodiments, the sign of effective treatment includes an improvement in one or more of (1) monocular best-corrected (BCVA) score from baseline in one or both eyes, (2) monocular low luminance visual acuity (LLVA) score from baseline in one or both eyes, (3) low luminance deficit (LLD) score from baseline in one or both eyes, as assessed by Early Treatment Diabetic Retinopathy Study (ETDRS) chart, e.g., as compared to untreated or placebo-treated subjects.

In some embodiments, the sign of effective treatment includes an increase in a National Eye Institute Visual Function Questionnaire, 25-item version (NEI VFQ-25) score from baseline. The NEI VFQ-25 (see Mangione et al., Arch Ophthalmol. 2001; 119(7):1050-1058; and Sivaprasad et al., Am J Ophthalmol. 2018; 190:1-8) measures the dimensions of self-reported vision-targeted health status of individuals with chronic eye conditions. The NEI VFQ-25 consists of 11 vision related domains: global vision rating, difficulty with near vision activities, difficulty with distance vision activities, limitations in social function related to vision, role limitations, dependency on others due to vision, mental health symptoms due to vision, driving difficulties, limitations with peripheral- and color-vision, and ocular pain.

The NEI VFQ-25 also includes a single item measuring general health. A composite score averages the vision related domains and ranges from 0 (worse) to 100 (best). The NEI VFQ 25 in patients with GA has been demonstrated to be a reliable and valid measure (see Sivaprasad et al. (2018), supra).

A representative NEI VFQ-25 questionnaire is shown in Table 2. A representative scoring key for the questionnaire is shown in Table 3. The averaging of items to generate VFQ-25 sub-scales is shown in Table 4.

TABLE 2
National Eye Institute Visual Function Questionnaire, 25-item version (NEI VFQ-25)
Part 1: General Health and Vision

| | | | | | | |
|---|---|---|---|---|---|---|
| 1. In general, would you say | Excellent | Very Good | Good | Fair | Poor | |
| your overall health is: | (1) | (2) | (3) | (4) | (5) | |
| 2. At the present time, | Excellent | Good | Fair | Poor | Very Poor | Completely Blind |
| would you say your eyesight | (1) | (2) | (3) | (4) | (5) | (6) |
| using both eyes (with | | | | | | |
| glasses or contact lenses, if | | | | | | |
| you wear them) is excellent, | | | | | | |
| good, fair, poor, or very | | | | | | |
| poor or are you complete | | | | | | |
| blind? | | | | | | |
| 3. How much of the time do | None of | A little of the | Some of the | Most of the | All of the | |
| you worry about your | the time | time | time | time | time | |
| eyesight? | (1) | (2) | (3) | (5) | (5) | |
| 4. How much pain or | None | Mild | Moderate | Severe | Very severe | |
| discomfort have you had in | (1) | (2) | (3) | (4) | (5) | |
| and around your eyes (for | | | | | | |
| example, burning, itching, or | | | | | | |
| aching)? | | | | | | |
| Would you say it is: | | | | | | |

Part 2: Difficulty with Activities

| | | | | | | |
|---|---|---|---|---|---|---|
| 5. How much difficulty do | No difficulty | A little | Moderate | Extreme | Stopped | Stopped doing this |
| you have reading ordinary | at all | difficulty | difficulty | difficulty | doing this | for other reasons |
| print in newspapers? | (1) | (2) | (3) | (4) | because of | or not interested in |
| Would you say you have: | | | | | your | doing this |
| | | | | | eyesight | (6) |
| | | | | | (5) | |
| 6. How much difficulty do | No difficulty | A little | Moderate | Extreme | Stopped | Stopped doing this |
| you have doing work or | at all | difficulty | difficulty | difficulty | doing this | for other reasons |
| hobbies that require you to | (1) | (2) | (3) | (4) | because of | or not interested in |
| see well up close, such as | | | | | your | doing this |
| cooking, sewing, fixing | | | | | eyesight | (6) |
| things around the house, or | | | | | (5) | |
| using hand tools? | | | | | | |
| Would you say: | | | | | | |
| 7. Because of your | No difficulty | A little | Moderate | Extreme | Stopped | Stopped doing this |
| eyesight, how much | at all | difficulty | difficulty | difficulty | doing this | for other reasons |
| difficulty do you have finding | (1) | (2) | (3) | (4) | because of | or not interested in |
| something on a crowded | | | | | your | doing this |
| shelf? | | | | | eyesight | (6) |
| | | | | | (5) | |
| 8. How much difficulty do | No difficulty | A little | Moderate | Extreme | Stopped | Stopped doing this |
| you have reading street | at all | difficulty | difficulty | difficulty | doing this | for other reasons |
| signs or the names of | (1) | (2) | (3) | (4) | because of | or not interested in |
| stores? | | | | | your | doing this |
| | | | | | eyesight | (6) |
| | | | | | (5) | |
| 9. Because of your | No difficulty | A little | Moderate | Extreme | Stopped | Stopped doing this |
| eyesight, how much | at all | difficulty | difficulty | difficulty | doing this | for other reasons |
| difficulty do you have going | (1) | (2) | (3) | (4) | because of | or not interested in |
| down steps, stairs, or curbs | | | | | your | doing this |
| in dim light or at night? | | | | | eyesight | (6) |
| | | | | | (5) | |
| 10. Because of your | No difficulty | A little | Moderate | Extreme | Stopped | Stopped doing this |
| eyesight, how much | at all | difficulty | difficulty | difficulty | doing this | for other reasons |
| difficulty do you have | (1) | (2) | (3) | (4) | because of | or not interested in |
| noticing objects off to the | | | | | your | doing this |
| side while you are walking | | | | | eyesight | (6) |
| along? | | | | | (5) | |
| 11. Because of your | No difficulty | A little | Moderate | Extreme | Stopped | Stopped doing this |
| eyesight, how much | at all | difficulty | difficulty | difficulty | doing this | for other reasons |
| difficulty do you have seeing | (1) | (2) | (3) | (4) | because of | or not interested in |
| how people react to things | | | | | your | doing this |
| you say? | | | | | eyesight | (6) |
| | | | | | (5) | |
| 12. Because of your | No difficulty | A little | Moderate | Extreme | Stopped | Stopped doing this |
| eyesight, how much | at all | difficulty | difficulty | difficulty | doing this | for other reasons |
| difficulty do you have | (1) | (2) | (3) | (4) | because of | or not interested in |
| picking out and matching | | | | | your | doing this |
| your own clothes? | | | | | eyesight | (6) |
| | | | | | (5) | |
| 13. Because of your | No difficulty | A little | Moderate | Extreme | Stopped | Stopped doing this |
| eyesight, how much | at all | difficulty | difficulty | difficulty | doing this | for other reasons |
| difficulty do you have | (1) | (2) | (3) | (4) | because of | or not interested in |
| visiting with people in their | | | | | your | doing this |
| homes, at parties, or in | | | | | eyesight | (6) |
| restaurants? | | | | | (5) | |
| 14. Because of your | No difficulty | A little | Moderate | Extreme | Stopped | Stopped doing this |
| eyesight, how much | at all | difficulty | difficulty | difficulty | doing this | for other reasons |
| difficulty do you have going | (1) | (2) | (3) | (4) | because of | or not interested in |
| out to see movies, plays, or | | | | | your | doing this |
| sports events? | | | | | eyesight | (6) |
| | | | | | (5) | |
| 15. Are you currently | Yes | No | | | | |
| driving, at least once in a | (1) | (2) | | | | |
| while? | Skip to Q 15c | | | | | |
| 15a. Have you never driven | Never | Gave up | | | | |
| a car or have you given up | drove | (2) | | | | |
| driving? | (1) | Skip to | | | | |
| | | Part 3, Q 17 | | | | |
| 15b. Was that mainly | Mainly | Mainly other | Both | | | |
| because of your eyesight | eyesight | reasons | eyesight and | | | |
| | (1) | (2) | other | | | |
| | Skip to | Skip to | reasons | | | |
| | Part 3, Q 17 | Part 3, Q 17 | (3) | | | |
| | | | Skip to | | | |
| | | | Part 3, Q 17 | | | |
| 15c. How much difficulty do | No difficulty | A little | Moderate | Extreme | | |
| you have driving during the | at all | difficulty | difficulty | difficulty | | |
| daytime in familiar places? | (1) | (2) | (3) | (4) | | |
| Would you say you have: | | | | | | |
| 16. How much difficulty do | No difficulty | A little | Moderate | Extreme | Stopped | Stopped doing this |
| you have driving at night? | at all | difficulty | difficulty | difficulty | doing this | for other reasons |
| Would you say you have: | (1) | (2) | (3) | (4) | because of | or not interested in |
| | | | | | your | doing this |
| | | | | | eyesight | (6) |
| | | | | | (5) | |
| 16a. How much difficulty do | No difficulty | A little | Moderate | Extreme | Stopped | Stopped doing this |
| you have driving in difficult | at all | difficulty | difficulty | difficulty | doing this | for other reasons |
| conditions, such as in bad | (1) | (2) | (3) | (4) | because of | or not interested in |
| weather, during rush hour, | | | | | your | doing this |
| on the freeway, or in city | | | | | eyesight | (6) |
| traffic? Would you say you | | | | | (5) | |
| have: | | | | | | |

Part 3: Responses to Vision Problems

| | | | | | |
|---|---|---|---|---|---|
| 17. Do you accomplish less | All of the | Most of the | Some of the | A little of | None of the |
| than you would like because | time | time | time | the time | time |
| of your vision? | (1) | (2) | (3) | (4) | (5) |
| 18. Are you limited in how | All of the | Most of the | Some of the | A little of | None of the |
| long you can work or do | time | time | time | the time | time |
| other activities because of | (1) | (2) | (3) | (4) | (5) |
| your vision? | | | | | |
| 19. How much does your | All of the | Most of the | Some of the | A little of | None of the |
| pain or discomfort in or | time | time | time | the time | time |
| around your eyes, for | (1) | (2) | (3) | (4) | (5) |
| example, burning, itching, or | | | | | |
| aching, keep you from doing | | | | | |
| what you'd like to be doing? | | | | | |
| Would you say: | | | | | |
| 20. I stay home most of the | Definitely | Mostly True | Not sure | Mostly False | Definitely |
| time because of my | True | (2) | (3) | (4) | False |
| eyesight. | (1) | | | | (5) |
| 21. I feel frustrated a lot of | Definitely | Mostly True | Not sure | Mostly False | Definitely |
| the time because of my | True | (2) | (3) | (4) | False |
| eyesight. | (1) | | | | (5) |
| 22. I have much less control | Definitely | Mostly True | Not sure | Mostly False | Definitely |
| over what I do, because of | True | (2) | (3) | (4) | False |
| my eyesight. | (1) | | | | (5) |
| 23. Because of my eyesight, | Definitely | Mostly True | Not sure | Mostly False | Definitely |
| I have to rely too much on | True | (2) | (3) | (4) | False |
| what other people tell me. | (1) | | | | (5) |
| 24. I need a lot of help from | Definitely | Mostly True | Not sure | Mostly False | Definitely |
| others because of my | True | (2) | (3) | (4) | False |
| eyesight. | (1) | | | | (5) |
| 25. I worry about doing | Definitely | Mostly True | Not sure | Mostly False | Definitely |
| things that will embarrass | True | (2) | (3) | (4) | False |
| myself or others, because of | (1) | | | | (5) |
| my eyesight, | | | | | |

TABLE 3
Scoring Key for NEI VFQ-25

| | Change original | To recoded |
|---|---|---|
| Item Number(s) | response category^{a} | value of: |
| 1, 3, 4, 15c^{b} | 1 | 100 |
| | 2 | 75 |
| | 3 | 50 |
| | 4 | 25 |
| | 5 | 0 |
| 2 | 1 | 100 |
| | 2 | 80 |
| | 3 | 60 |
| | 4 | 40 |
| | 5 | 20 |
| | 6 | 0 |
| 5-14, 16, 16a | 1 | 100 |
| | 2 | 75 |
| | 3 | 50 |
| | 4 | 25 |
| | 5 | 0 |
| | 6 | * |
| 17-25 | 1 | 0 |
| | 2 | 25 |
| | 3 | 50 |
| | 4 | 75 |
| | 5 | 100 |

^{a}Precoded response choices as printed in the questionnaire.
^{b}Item 15c has four-response levels, but is expanded to five levels using item 15b. Of 15b = 1, then 15c should be recorded to “0.” If 15b = 2, then 15c should be recorded to missing. If 15b = 3, then 15c should be recoded to missing.
* Response choice “6” indicates that the person does not perform the activity because of non-vision related problems. If this choice is selected, the item is coded as “missing.”

TABLE 4
Averaging of Items to Generate VFQ-25 Sub-Scales

| | | Items to be Averaged (after |
|---|---|---|
| Scale | Number of Items | recoding per Table 3) |
| General Health | 1 | 1 |
| General Vision | 1 | 2 |
| Ocular Pain | 2 | 4, 19 |
| Near Activities | 3 | 5, 6, 7 |
| Distance Activities | 3 | 8, 9, 14 |
| Vision Specific: | | |
| Social Functioning | 2 | 11, 13 |
| Mental Health | 4 | 3, 21, 22, 25 |
| Role Difficulties | 2 | 17, 18 |
| Dependency | 3 | 20, 23, 24 |
| Driving | 3 | 15c, 16, 16a |
| Color Vision | 1 | 12 |
| Peripheral Vision | 1 | 10 |

The EQ-5D-5L is a standardized questionnaire for measuring health-related quality of life and is defined in 5 dimensions, I.e., mobility, usual activities, self-care, pain/discomfort, and anxiety/depression. A 0 to 100 health state visual analog scale (VAS) accompanies the above 5 dimensions, where 0 indicates worst health and 100 best health. A 0 to 1 index or utility score is calculated from the 5 dimensions using a preference-based value set, where 0 indicates a health state equivalent to death and 1 indicates perfect health. Negative values indicate health states considered worse than death. A EQ-5D-5L questionnaire is provided in Table 5 below.

TABLE 5
EuroQol 5-Dimension 5-Level Questionnaire (EQ-5D-5L)
MOBILITY

| | |
|---|---|
| I have no problems in walking about | 1 |
| I have no slight problems in walking about | 2 |
| I have moderate problems in walking about | 3 |
| I have severe problems in walking about | 4 |
| I am unable to walk about | 5 |

SELF-CARE

| | |
|---|---|
| I have no problems washing or dressing myself | 1 |
| I have slight problems washing or dressing myself | 2 |
| I have moderate problems washing or dressing myself | 3 |
| I have severe problems washing or dressing myself | 4 |
| I am unable to wash or dress myself | 5 |

USUAL ACTIVITIES (e.g., work, study,
housework, family or leisure activities)

| | |
|---|---|
| I have no problems doing my usual activities | 1 |
| I have slight problems doing my usual activities | 2 |
| I have moderate problems doing my usual activities | 3 |
| I have severe problems doing my usual activities | 4 |
| I am unable to do my usual activities | 5 |

PAIN/DISCOMFORT

| | |
|---|---|
| I have no pain or discomfort | 1 |
| I have slight pain or discomfort | 2 |
| I have moderate pain or discomfort | 3 |
| I have severe pain or discomfort | 4 |
| I have extreme pain or discomfort | 5 |

ANXIETY/DEPRESSION

| | |
|---|---|
| I am not anxious or depressed | 1 |
| I am slightly anxious or depressed | 2 |
| I am moderately anxious or depressed | 3 |
| I am severely anxious or depressed | 4 |
| I am extremely anxious or depressed | 5 |

In some embodiments, the treatment of GA secondary to AMD includes an improvement in a EuroQoL 5-dimension 5-level questionnaire (EQ-5D-5L) score in one or more of mobility, usual activities, self-care, pain/discomfort, and anxiety/depression from baseline. The improvement may be a reduction a decrease in an EQ-5D-5L score in one or more mobility, usual activities, self-care, pain/discomfort, and anxiety/depression from baseline, e.g., as compared to a control (such as an untreated or a placebo-treated subject). The improvement may also be an increase in an EQ-5D-5L score in one or more mobility, usual activities, self-care, pain/discomfort, and anxiety/depression from baseline, e.g., as compared to a control (such as an untreated or a placebo-treated subject).

The Lawton Instrumental Activities of Daily Living (IADL) is an assessment that evaluates the patient's ability for independent living (see Lawton et al., Gerontologist. 1969; 9(3):179-186). The IADL is used to capture the ability in functioning at the present time and in evaluating functioning improvements or deterioration over time. The Lawton IADL scale (representative scale shown in Table 6) consists of 8 domains of functioning (food preparation, housekeeping, laundry, ability to use the telephone, mode of transportation, shopping, financial, and medication management). The instrument includes 8 dichotomous questions and its total score can range from 0 to 8. Low scores depict low function and dependence, whereas high scores depict high function and independence. An IADL scale is provided in Table 6 below.

TABLE 6
Lawton Instrumental Activities of Daily Living (IADL) Scale
Ability to Use Telephone

| | |
|---|---|
| 1. Operates telephone on own initiative; looks up | 1 |
| and dials numbers | |
| 2. Dials a few well-known numbers | 1 |
| 3. Answers telephone, but does not dial | 1 |
| 4. Does not use telephone at all | 0 |

Shopping

| | |
|---|---|
| 1. Takes care of all shopping needs independently | 1 |
| 2. Shops independently for small purchases | 0 |
| 3. Needs to be accompanied on any shopping trip | 0 |
| 4. Completely unable to shop | 0 |

Food Preparation

| | |
|---|---|
| 1. Plans, prepares, and serves adequate meals | 1 |
| independently | |
| 2. Prepares adequate meals if supplied with | 0 |
| ingredients | |
| 3. Heats and serves prepared meals or prepares | 0 |
| meals but does not maintain adequate | |
| 4. Needs to have meals prepared and served | 0 |

Housekeeping

| | |
|---|---|
| 1. Maintains house alone with occasion assistance | 1 |
| (heavy work) | |
| 2. Performs light daily tasks such as dishwashing, | 1 |
| bed making | |
| 3. Performs light daily tasks, but cannot maintain | 1 |
| acceptable level of cleanliness | |
| 4. Needs help with all home maintenance tasks | 1 |
| 5. Does not participate in any housekeeping tasks | 0 |

Laundry

| | |
|---|---|
| 1. Does personal laundry completely | 1 |
| 2. Launders small items, rinses socks, stocking, | 1 |
| etc. | |
| 3. All laundry must be done by others | 0 |

Mode of Transportation

| | |
|---|---|
| 1. Travels independently on public transportation | 1 |
| or drives own car | |
| 2. Arranges own travel via taxi, but does not | 1 |
| otherwise use public transportation | |
| 3. Travels on public transportation when assisted | 1 |
| or accompanied by another | |
| 4. Travel limited to taxi or automobile with | 0 |
| assistance of another | |
| 5. Does not travel at all | 0 |

Responsibility for Own Medications

| | |
|---|---|
| 1. Is responsible for taking medication in correct | 1 |
| dosages at correct time | |
| 2. Takes responsibility if medication is prepared in | 0 |
| advance in separate dosages | |
| 3. Is not capable of dispensing own medication | 0 |

Ability to Handle Finances

| | |
|---|---|
| 1. Manages financial matters independently | 1 |
| (budgets, writes checks, pays rent and bills, goes | |
| to bank); collects and keeps track of income | |
| 2. Manages day-to-day purchases, but needs help | 1 |
| with banking, major purchases, etc. | |
| 3. Incapable of handling money | 0 |

In some embodiments, the treatment of GA secondary to AMD includes an improvement in a Lawton IADL score (by 1, 2, 3, 4, 5, 6, 7, or 8 points) from baseline, e.g., as compared to a control (such as an untreated or a placebo-treated subject). The improvement may be a reduction a decrease in the Lawton IADL score from baseline, e.g., as compared to a control (such as an untreated or a placebo-treated subject). The improvement may also be an increase in the Lawton IADL from baseline, e.g., as compared to a control (such as an untreated or a placebo-treated subject).

In some embodiments, the treatment of GA secondary to AMD includes an improvement in reading speed from baseline, e.g., as compared to a control (such as an untreated or a placebo-treated subject). In some embodiments, the monocular and/or binocular reading speed is assessed by MNRead Acuity Charts Radner Reading Charts.

The MNRead acuity cards consist of single, simple sentences with equal numbers of characters. The print is a proportionally spaced font, similar to that found in many newspapers and books. The cards contain sentences with 19 different print sizes. The text is printed with high contrast (approximately 85%). Each sentence contains 60 characters (including space between each word and at the end of each line) printed as three lines with even left and right margins. The vocabulary used in the sentences is selected from words appearing with high frequency in second- to third-grade reading materials (see Calabrese et al., JAMA Ophthalmology. 2016; 134(4):398-405).

The Radner Reading Cards (see Radner W., Graefes Arch Clin Exp Ophthalmol. 2017; 255(8): 1465-1482) can be used to measure the reading speed if MNRead card is not available. The test consists of 24 short sentences that are highly comparable in terms of number of words, word length, position of words, lexical difficulty, and syntactical complexity.

The present disclosure also provides methods for treating iAMD with Compound 1 or a pharmaceutically acceptable salt, which reduce the risk of iAMD in a subject (in one or both eyes) converting to late AMD (e.g., iRORA to cRORA, high-risk drusen to late AMD, or iAMD to late AMD). The clinical classifications of AMD are provided in Table 1.

### Pharmaceutical Compositions

The disclosure also relates to use of pharmaceutical compositions including Compound 1 and or a pharmaceutically acceptable salt thereof and a pharmaceutically acceptable excipient. Any suitable pharmaceutical compositions and formulations, as well as suitable methods for formulating and suitable routes and suitable sites of administration, are within the scope of this disclosure. Also, unless otherwise stated, any suitable dosage(s) and frequency of administration are contemplated.

Unless otherwise noted, the dosage level of Compound 1 or a pharmaceutically acceptable salt thereof can be any suitable level. In some embodiments, the dosage levels of Compound 1 or a pharmaceutically acceptable salt thereof for a subject can generally be between about 1 mg/kg and about 100 mg/kg (e.g., between about 2 mg/kg and about 50 mg/kg, between about 5 mg/kg and about 25 mg/kg), per treatment.

A suitable dose of Compound 1 or a pharmaceutically acceptable thereof which is capable of treating GA secondary to AMD (or treating iAMD) in a subject, can depend on a variety of factors including, e.g., the age, gender, and weight of a subject to be treated and the particular inhibitor compound used. Other factors affecting the dose administered to the subject include, e.g., the severity of the condition to be treated. Other factors can include, e.g., other medical disorders concurrently or previously affecting the subject, the general health of the subject, the genetic disposition of the subject, diet, time of administration, rate of excretion, drug combination, and any other additional therapeutics that are administered to the subject. It should also be understood that a specific dosage and treatment regimen for any particular subject will depend upon the judgment of the treating medical practitioner (e.g., doctor or nurse). A pharmaceutical composition can include a therapeutically effective amount of Compound 1 or a pharmaceutically acceptable salt thereof.

The compositions can be administered to a human subject using a variety of methods that depend, in part, on the route of administration. The route can be, e.g., oral, sublingual, buccal, transdermal, intradermal, intramuscular, parenteral, intravenous, intra-arterial, intracranial, subcutaneous, ophthalmic, intraventricular, intraspinal, intraperitoneal, intranasal, inhalation, and topical administration.

### Formulations for Oral Administration

In some embodiments, a composition is formulated for oral administration (“oral dosage forms”). Oral dosage forms can be, for example, in the form of tablets, capsules, a liquid solution or suspension, a powder, or liquid or solid crystals, which contain the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like. Compositions for oral administration may also be presented as chewable tablets, as hard gelatin capsules where the active ingredient is mixed with an inert solid diluent (e.g., potato starch, lactose, microcrystalline cellulose, calcium carbonate, calcium phosphate or kaolin), or as soft gelatin capsules where the active ingredient is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment.

Controlled release compositions for oral use may be constructed to release the active drug by controlling the dissolution and/or the diffusion of the active drug substance. Any of a number of strategies can be pursued in order to obtain controlled release and the targeted plasma concentration versus time profile. In one example, controlled release is obtained by appropriate selection of various formulation parameters and ingredients, including, e.g., various types of controlled release compositions and coatings. Examples include single or multiple unit tablet or capsule compositions, oil solutions, suspensions, emulsions, microcapsules, microspheres, nanoparticles, patches, and liposomes. In some embodiments, compositions include biodegradable, pH, and/or temperature-sensitive polymer coatings.

Dissolution or diffusion-controlled release can be achieved by appropriate coating of a tablet, capsule, pellet, or granulate formulation of compounds, or by incorporating the compound into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, di-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

The liquid forms in which compositions can be incorporated for administration orally include aqueous solutions, suitably flavored syrups, aqueous or oil suspensions, and flavored emulsions with edible oils, e.g., cottonseed oil, sesame oil, coconut oil, or peanut oil, as well as elixirs and similar pharmaceutical vehicles.

### Kits and Unit Dosage Forms

Also provided herein are kits that include Compound 1 or a pharmaceutically acceptable salt thereof in a therapeutically effective amount (e.g., in a pharmaceutical composition) for use in any one or more of the methods disclosed herein. The kit may optionally include instructions, e.g., comprising administration schedules, to allow a practitioner (e.g., a physician, nurse, or patient) to administer Compound 1 or the pharmaceutically acceptable salt thereof, e.g., in a pharmaceutical composition further including a pharmaceutically acceptable carrier) contained therein to a patient. The kit may further include a syringe.

Kits can optionally include multiple packages of the single-dose pharmaceutical compositions each containing an effective amount of Compound 1 or a pharmaceutically acceptable salt thereof e.g., in a pharmaceutical composition) for a single administration in accordance with the methods provided above. Instruments or devices for administering Compound 1 or a pharmaceutically acceptable salt thereof (e.g., in a pharmaceutical composition) may also be included in the kits. A kit may provide one or more pre-filled syringes containing an effective amount of Compound 1 or a pharmaceutically acceptable salt thereof e.g., in a pharmaceutical composition).

The following examples are merely illustrative and should not be construed as limiting the scope of this disclosure in any way as many variations and equivalents will become apparent to those skilled in the art upon reading the present disclosure. The contents of all references, accessioned entries (e.g., PUBMED, GENBANK, UNIPROT, PUBCHEM entries), patents, and patent applications cited throughout this application are expressly incorporated herein by reference.

### EXAMPLES

### Example 1. A Phase 2, Double-Masked, Placebo-Controlled, Dose Range Finding Study of Danicopan (ALXN2040) in Patients with Geographic Atrophy (GA) Secondary to Age-Related Macular Degeneration (AMD)

A multicenter Phase 2, randomized, double-masked, placebo-controlled, dose-finding, parallel-group study is conducted to evaluate the efficacy, safety, and pharmacokinetics (PK) of danicopan compared to placebo in patients ≥60 years with GA secondary to AMD.

### Objectives and Endpoints

The objectives and corresponding endpoints are summarized in Table 7 below.

TABLE 7
Study Objectives and Endpoints

| | |
|---|---|
| Objectives | Endpoints |

Primary

| | |
|---|---|
| To evaluate the effect of different dosage regimens | Change from Baseline at Week 52 in the |
| of danicopan on the progression of GA secondary | square root (sqrt) of total GA lesion area (mm) |
| to AMD compared to placebo | in the study eye* as measured by fundus |
| | autofluorescence (FAF) |
| | *All patients will undergo monocular test on both |
| | eyes. The study eye is the eye that meets eligibility |
| | criteria. If both eyes are eligible, the right eye is |
| | taken as the study eye. |

Secondary

| | |
|---|---|
| To evaluate the effect of danicopan on disease | Change from Baseline to Week 104 in the sqrt |
| progression utilizing anatomical measures in the | of the total GA lesion area (mm) in the study |
| study eye | eye as measured by FAF |
| | Change from Baseline to Week 52 and Week |
| | 104 in the total GA lesion area (mm²) in the |
| | study eye as measured by FAF |
| To evaluate the effect of danicopan on disease | Change from Baseline to Week 52 and Week |
| progression utilizing functional measures in the | 104 in monocular best-corrected visual acuity |
| study eye | (BCVA) scores in the study eye as assessed |
| | by Early Treatment Diabetic Retinopathy Study |
| | (ETDRS) chart |
| | Change from Baseline to Week 52 and Week |
| | 104 in monocular low luminance visual acuity |
| | (LLVA) scores in the study eye as assessed by |
| | ETDRS chart |
| | Change from Baseline to Week 52 and Week |
| | 104 in low luminance deficit (BCVA-LLVA) in |
| | the study eye |
| | Change from Baseline to Week 52 and Week |
| | 104 in monocular reading speeds in the study |
| | eye as assessed by Minnesota Low Vision |
| | Reading Test (MNRead) Acuity Charts or |
| | Radner Reading Charts |
| To evaluate the effect of danicopan on patient | Change from Baseline to Week 52 and Week |
| reported outcomes (PROs) in patients with GA | 104 in National Eye Institute Visual Function |
| secondary to AMD | Questionnaire (NEI VFQ-25) scores |
| To evaluate the PK and pharmacodynamics (PD) of | Plasma concentrations of danicopan over time |
| danicopan in patients with GA secondary to AMD | PD biomarkers, ex vivo serum AP activity, and |
| | plasma Bb concentration over time |
| To evaluate the safety and tolerability of danicopan | Incidence of treatment-emergent adverse |
| in patients with GA secondary to AMD | events (TEAEs), serious adverse events |
| | (SAEs), and ocular TEAEs, SAEs, and clinical |
| | laboratory abnormalities, and events leading to |
| | discontinuation of study drug throughout the |
| | study |

Exploratory

| | |
|---|---|
| To evaluate the effect of danicopan on health- | Change from Baseline to Week 52 and Week |
| related quality of life and activities of daily living | 104 in EQ-5D-5L |
| | Change from Baseline to Week 52 and Week |
| | 104 in Lawton Instrumental Activities of Daily |
| | Living (IADL) |
| To evaluate the effect of danicopan on disease | Change from Baseline to Week 52 and Week |
| progression utilizing exploratory anatomical | 104 in the sqrt of the total GA lesion area (mm) |
| measures | in fellow eye and both eyes as measured by |
| | FAF |
| | Change from Baseline to Week 52 and Week |
| | 104 in the total GA lesion area (mm2) in fellow |
| | eye and both eyes as measured by FAF |
| | Percent change from Baseline to Week 52 and |
| | Week 104 in the total GA lesion area (mm²) in |
| | the study eye, fellow eye and both eyes |
| | combined as measured by FAF |
| | Percent change from Baseline to Week 52 and |
| | Week 104 in the sqrt of the total GA lesion area |
| | (mm) in the study eye, fellow eye and both |
| | eyes combined as measured by FAF |
| | Incidence of patients with conversion from |
| | incomplete to complete retinal pigment |
| | epithelium and outer retinal atrophy (iRORA to |
| | CRORA) from Baseline to Week 52 and Week |
| | 104 in the study eye, fellow eye, either eye, |
| | and both eyes combined using spectral domain |
| | optical coherence tomography (SD OCT) |
| | images |
| | Incidence of patients with conversion from |
| | high-risk drusen to late AMD from Baseline to |
| | Week 52 and Week 104 in in the study eye, |
| | fellow eye, either eye, and both eyes combined |
| | using SD OCT images |
| | Incidence of patients with conversion from |
| | intermediate AMD (iAMD) to late AMD from |
| | Baseline to Week 52 and Week 104 in the |
| | study eye, fellow eye, either eye, and both |
| | eyes combined using SD-OCT images |
| | Number of conversions from iAMD to late AMD |
| | from Baseline in the study eye, fellow eye, and |
| | both eyes combined to Week 52 and Week 104 |
| | Change from Baseline to Week 52 and Week |
| | 104 in drusen volume (mm³) in the fellow eye |
| | with early or iAMD as measured by SD OCT |
| | Change from Baseline to Week 52 and Week |
| | 104 in total GA lesion area (mm²) and sqrt of |
| | the total GA lesion area (mm) in the study eye, |
| | the fellow eye, and both eyes combined as |
| | measured by SD OCT |
| | Percent change from Baseline to Week 52 and |
| | Week 104 in the total GA lesion area (mm²) |
| | and sqrt of the total GA lesion area (mm) in |
| | study eye, fellow eye and both eyes combined |
| | as measured by SD-OCT |
| To evaluate the effects of danicopan on disease | Change from Baseline to Week 52 and Week |
| progression utilizing exploratory functional | 104 in BCVA scores in the fellow eye as |
| measures | assessed by ETDRS chart |
| | Change from Baseline to Week 52 and Week |
| | 104 in LLVA scores in the fellow eye as |
| | assessed by ETDRS chart |
| | Change from Baseline to Week 52 and Week |
| | 104 in low luminance deficit (BCVA-LLVA) in |
| | the fellow eye |
| | Change from Baseline to Week 52 and Week |
| | 104 in binocular BCVA scores as assessed |
| | ETDRS chart |
| | Change from Baseline to Week 52 and Week |
| | 104 in binocular LLVA scores as assessed by |
| | ETDRS chart |
| | Change from Baseline to Week 52 and Week |
| | 104 in binocular low luminance deficit (BCVA- |
| | LLVA) |
| | Change from Baseline to Week 52 and Week |
| | 104 in monocular reading speeds in fellow eye |
| | as assessed by MNRead Acuity Charts or |
| | Radner Reading Charts |
| | Change from Baseline to Week 52 and Week |
| | 104 in binocular reading speeds as assessed |
| | by MNRead Acuity Charts or Radner Reading |
| | Charts |
| | Number of scotomatous points assessed by |
| | mesopic microperimetry for the evaluation of |
| | the macular functional response in study eye |
| | and fellow eye to Week 52 and Week 104^{a} |
| | Change from Baseline in macular sensitivity as |
| | assessed by mesopic microperimetry in study |
| | eye and fellow eye to Week 52 and Week 104 |
| To evaluate the effect of optimal danicopan dose | Change from Baseline at Week 52 and at |
| on disease progression at the end of delayed-start | Week 104 in the sqrt of the total GA lesion area |
| period (Week 104) compared to the end of early- | (mm) in the study eye as measured by FAF |
| start period (Week 52) for the study eye | |
| To evaluate exploratory PD biomarkers | Concentrations of exploratory biomarkers over |
| | time, including serum CP activity and serum (or |
| | plasma) C3 and FD concentrations |

Error! Reference source not found.
Microperimetry endpoints will be assessed in the microperimetry subpopulation
Abbreviations: AMD = age-related macular degeneration; AP = alternative pathway; Bb = Bb fragment of complement factor B; BCVA = best-corrected visual acuity; C3 = complement component 3; CP = classical pathway; cRORA = complete retinal pigment epithelium and outer retinal atrophy; EQ-5D-5L = EuroQol 5-dimension 5-level questionnaire; ETDRS = Early Treatment Diabetic Retinopathy Study; FAF = fundus autofluorescence; FD = factor D; GA = geographic atrophy; IADL = Instrumental Activities of Daily Living; iAMD = intermediate age-related macular degeneration; iRORA = incomplete retinal pigment epithelium and outer retinal atrophy; LLVA = low luminance visual acuity; MNRead = Minnesota Low Vision Reading Test; NEI VFQ-25 = National Eye Institute Visual Function Questionnaire, 25-item version; PD = pharmacodynamic; PK = pharmacokinetic; PRO = patient reported outcome; SAE = serious adverse event; sqrt = square root; SD-OCT = spectral-domain optical coherence tomography; TEAE = treatment-emergent adverse event

### Overall Design

This is a multicenter Phase 2, randomized, double-masked, placebo-controlled, dose-finding, parallel-treatment study to evaluate the efficacy and safety, and PK of danicopan compared to placebo in patients ≥60 years with GA secondary to AMD. Eligible patients will be randomized (1:1:1:1) with stratification to 1 of 4 treatment groups (3 active treatment groups and 1 placebo group). The primary efficacy analysis will be performed when all patients complete the Week 52 visit or discontinued Secondary efficacy and safety analyses, and exploratory analyses will be performed when all patients complete the Week 104 visit or discontinue.

There are 2 potential interim analyses (IA) planned for this study. The first interim analysis (IA1) for futility may be conducted when approximately 50% patients have completed the Week 28 visit. The second interim analysis (IA2) may be conducted when approximately 50% of patients complete Week 52 visit. At IA2, a futility analysis will be conducted first. If the study is not considered futile, dose response analysis will be performed. If the dose response analysis is positive, a pair-wise comparison of each dose compared to placebo will be conducted.

A dose with the optimal benefit-risk profile could be identified at the IA2 or primary analysis for Phase 3 development. Placebo patients will be re-randomized to one of the 3 active treatment groups at Week 52, or switched to the optimal dose, if already identified. If an optimal dose is identified, all patients who have at least 52 weeks of treatment on their originally assigned dose will be switched to the selected optimal dose for the remainder of the study. Masked treatment paradigm will be maintained throughout the study.

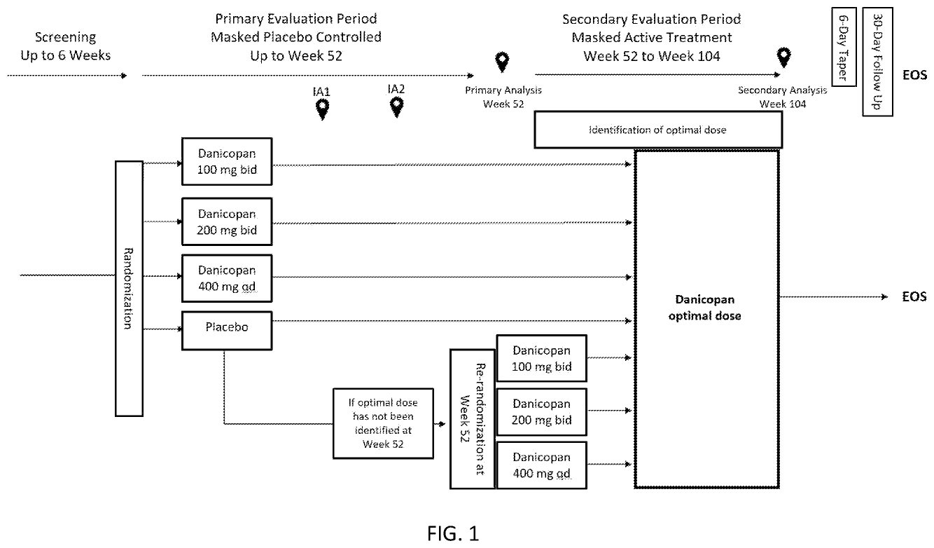

Approximately 332 patients ≥60 years will be enrolled, with 83 patients per dose group. The four treatment arms are: (1) 100 mg 2× daily (BID) dose group, (2) 200 mg BID dose group, (3) 400 once daily (QD) dose group, and (4) placebo group. The study consists of a Screening Period of up to 6 weeks and a Masked Treatment Period (approximately 2 years) (FIG. 1). A 30-day follow-up is scheduled after the last dose. The total study duration per patient is approximately 166 weeks. Treatment ends with a 6-day Taper, followed by a Follow-up Visit 30 days after the last taper dose. The potential total study duration per patient is approximately 115 weeks. The Schedule of Activities (SoA) is summarized in Tables 8-10 below.

TABLE 8
Schedule of Activities for Screening and the Primary Evaluation Period (Up to Week 52)

| | Screening | Primary Evaluation Period (Masked Treatment) | | | | | | | | |
|---|---|---|---|---|---|---|---|---|---|---|
| | Visit | | | | | | | | | |
| | Up to 6 | | | | | | | | | Notes |
| | weeks | 1 | 2 | 3 | 4 | 5 | 6 | 7 | 8 | X = in-clinic visit; |
| | Weeks | | | | | | | | | HV = home visit by VHS; |
| | −6 to 0 | | 2 | 4 | 8 | 16 | 28 | 40 | 52 | HV may be turned into in- |
| | Days and Window | | | | | | | | | clinic visit per agreement |
| | −42 to −1 | 1 | 15 ± 2 | 29 ± 4 | 57 ± 4 | 113 ± 4 | 197 ± 4 | 281 ± 4 | 365 ± 4 | between patient and site |
| | Visit Type | | | | | | | | | but following the same HV |
| Procedure | X | X | HV | X | X | X | X | X | X | assessments. |
| Informed consent | X | | | | | | | | | To be obtained before any |
| | | | | | | | | | | study-related procedures |
| | | | | | | | | | | are performed |
| Inclusion and exclusion | X | X | | | | | | | | Eligibility to be confirmed |
| criteria | | | | | | | | | | prior to randomization. |
| Randomization | | X | | | | | | | | |
| Demography | X | | | | | | | | | |
| Full PE including height | X | X | | | X | | | | X | Height only required at |
| and weight | | | | | | | | | | Screening |
| Abbreviated PE | | | | X | | X | X | X | | Abbreviated PE = a |
| | | | | | | | | | | targeted PE that includes, |
| | | | | | | | | | | at a minimum, body system |
| | | | | | | | | | | relevant examination |
| | | | | | | | | | | based on Investigator |
| | | | | | | | | | | judgement and patient |
| | | | | | | | | | | symptoms |
| Vital signs | X | X | X | X | X | X | X | X | X | |
| Medical history | X | | | | | | | | | |
| Prior and current | X | X | | | | | | | | |
| medications | | | | | | | | | | |
| FSH for females | X | | | | | | | | | |
| Screening tests for | X | | | | | | | | | |
| HIV, HBV, HCV | | | | | | | | | | |
| Vaccination or | X | continuous monitoring | | | | | | | | |
| confirmation of | | | | | | | | | | |
| vaccination against | | | | | | | | | | |
| Neisseria meningitidis | | | | | | | | | | |
| Laboratory | X | X | X | X | X | X | X | X | X | VHS will collect blood |
| assessments (including | | | | | | | | | | samples at HV. |
| chemistry, hematology, | | | | | | | | | | |
| urinalysis, and LFTs) | | | | | | | | | | |
| PK | | X | | X | | X | X | X | X | PK samples will be |
| | | | | | | | | | | collected predose, and 2 h |
| | | | | | | | | | | postdose for all visits |
| | | | | | | | | | | except for Week 4. For |
| | | | | | | | | | | Week 4, PK samples |
| | | | | | | | | | | should be collected on |
| | | | | | | | | | | predose, 2 h, and 4 h post |
| | | | | | | | | | | dose. |
| PD (Bb and AP activity) | | X | | X | | X | X | X | X | AP activity samples will be |
| | | | | | | | | | | collected at the same time |
| | | | | | | | | | | points as PK samples; Bb |
| | | | | | | | | | | samples will be collected at |
| | | | | | | | | | | predose; at ET; Bb and AP |
| | | | | | | | | | | activity samples will be |
| | | | | | | | | | | collected once. |
| Biomarkers | | X | | X | | X | X | X | X | Biomarkers will be |
| | | | | | | | | | | collected predose and also |
| | | | | | | | | | | once at ET |
| Genetic samples | | X | | | | | | | | |
| (optional) | | | | | | | | | | |
| 12-lead ECG | | X | | | | | | | X | Single tracing |
| Drug dispensing | | X | | X | X | X | X | X | X | At each clinic visit, site |
| | | | | | | | | | | should ensure patient has |
| | | | | | | | | | | sufficient study drug till |
| | | | | | | | | | | their next visit |
| Drug accountability | | | X | X | X | X | X | X | X | Patients are instructed to |
| | | | | | | | | | | bring their study drugs at |
| | | | | | | | | | | each clinic visit |
| AE/SAE monitoring | continuous monitoring | | | | | | | | | Continuous monitoring of |
| | | | | | | | | | | AE/SAEs (including |
| | | | | | | | | | | pregnancy in female |
| | | | | | | | | | | partners of male patients) |
| | | | | | | | | | | starts at time of signing the |
| | | | | | | | | | | ICF and continues until |
| | | | | | | | | | | EOS |
| Concomitant | continuous monitoring | | | | | | | | | Continuous monitoring |
| medications monitoring | | | | | | | | | | (reporting of concomitant |
| | | | | | | | | | | medications) starts at time |
| | | | | | | | | | | of signing the ICF and |
| | | | | | | | | | | continues until EOS |
| Review patient safety | continuous monitoring | | | | | | | | | Patients to carry card at all |
| card | | | | | | | | | | times |
| PATIENT-REPORTED OUTCOMES | | | | | | | | | | All PROs and QoL |
| | | | | | | | | | | assessments should be |
| NEI VFQ-25 | | X | | | X | | X | X | X | completed as early as |
| EQ-5D-5L | | X | | | X | | X | X | X | possible during visits, |
| Lawton IADL | | X | | | X | | X | X | X | before ocular assessments |
| OCULAR ASSESSMENTS | | | | | | | | | | Ocular assessments are to |
| | | | | | | | | | | be performed on both eyes |
| | | | | | | | | | | (OU); tests that do not |
| | | | | | | | | | | need pupil dilation should |
| | | | | | | | | | | be done first |
| BCVA testing on | X | X | | | X | X | X | X | X | Non-dilated, to be |
| ETDRS chart (starting | | | | | | | | | | administered first |
| at 4 m) | | | | | | | | | | monocularly (right/left eye) |
| | | | | | | | | | | and then binocularly (both |
| | | | | | | | | | | eyes). |
| LL BCVA testing on | | X | | | | | X | | X | Non-dilated, to be |
| ETDRS chart (starting | | | | | | | | | | administered first |
| at 4 m) | | | | | | | | | | monocularly (right/left eye) |
| | | | | | | | | | | and then binocularly (both |
| | | | | | | | | | | eyes). |
| Reading Speed | | X | | | | | X | | X | Non-dilated, to be |
| (MNRead or Radner | | | | | | | | | | administered first |
| Reading Charts)^{a} | | | | | | | | | | monocularly (right/left eye) |
| | | | | | | | | | | and then binocularly (both |
| | | | | | | | | | | eyes). |
| Mesopic | X^{c} | | | | | | X | | X | Non-dilated, only in |
| microperimetry^{b} | | | | | | | | | | microperimetry |
| | | | | | | | | | | subpopulation |
| Slit lamp examination | X | X | | | X | X | X | X | X | |
| Tonometry/IOP | X | X | | | X | X | X | X | X | Goldmann applanation |
| | | | | | | | | | | tonometry must be used at |
| | | | | | | | | | | Screening. Tono-pen or |
| | | | | | | | | | | other calibrated tonometers |
| | | | | | | | | | | can be used at other times. |
| | | | | | | | | | | Goldmann applanation |
| | | | | | | | | | | tonometry must also be |
| | | | | | | | | | | used to verify the reading |
| | | | | | | | | | | of ≥30 mmHg occurring at |
| | | | | | | | | | | any time. |
| Dilated binocular | X | X | | | X | X | X | X | X | |
| indirect | | | | | | | | | | |
| ophthalmoscopy | | | | | | | | | | |
| FAF | X | X | | | X | X | X | X | X | Recommended order of |
| NIR | X | X | | | X | X | X | X | X | imaging is FAF, NIR, SD- |
| SD-OCT | X | X | | | X | X | X | X | X | OCT, FA, CFP. |
| Fluorescein | X | | | | | | X | | X | Administration of artificial |
| angiography | | | | | | | | | | tears between acquisition |
| CFP | X | | | | | | X | | X | of each eye for FAF/NIR |
| Biometry (if available) ^{d} | X | | | | | | | | | and SD-OCT is |
| | | | | | | | | | | recommended. |

^{a}Reading speeds will only be assessed if charts are available in the local language.
^{b}Microperimetry should be performed prior to any imaging.
^{c}Microperimetry should be carried out twice per eye during Screening.
^{d} Biometry should be performed if the site has the equipment; otherwise, spherical equivalent refractive error should be determined.
Abbreviations:
AE = adverse event; AP = alternative pathway; Bb = Bb fragment of complement factor B; BCVA = best-corrected visual acuity; CFP = color fundus photography; ECG = electrocardiogram; ETDRS = Early Treatment Diabetic Retinopathy Study; EOS = end of study; EQ-5D -5L = EuroQol 5-dimension 5-level questionnaire; ET = early termination; FA = fluorescein angiography; FAF = fundus autofluorescence; FSH = follicle stimulating hormone; h = hour; HBV = hepatitis B virus; HCV = hepatitis C virus; HIV = human immunodeficiency virus; HV = home visit; IADL = Instrumental Activities of Daily Living; ICF = informed consent form; IOP = intraocular pressure; LFT = liver function test; LL BCVA = low luminance best-corrected visual acuity; MNRead = Minnesota Low-Vision Reading Test; NEI VFQ-25 = National Eye Institute Visual Function Questionnaire, 25-item version; NIR = near infrared reflectance; OU = oculus uterque/both eyes; PD = pharmacodynamic; PE = physical examination; PK = pharmacokinetics; PRO = patient-reported outcome; QoL = quality of life; SAE = serious adverse event; SD-OCT = spectral domain optical coherence tomography; VHS = visiting health service

TABLE 9
Schedule of Activities for the Secondary Evaluation Period (Week 52 to Week 104) and Optimal Dose Transition

| | | | | | | | | Optimal Dose | | | | |
|---|---|---|---|---|---|---|---|---|---|---|---|---|
| | | | | | | | | Transition^{a} | | | | |
| | Secondary Evaluation Period (Masked Treatment) Weeks 52 to 104 | | | | | | | After Week 52 | | | | |
| | Visit | | | | | | | | | | | |
| | 9 | 10 | 11 | 12 | 13 | 14 | 15 | | | | | Notes |
| | Weeks | | | | | | | | | | | X = in-clinic visit; |
| | | | | | | | | Optimal | | | | HV = home visit by VHS |
| | 54 | 56 | 60 | 68 | 80 | 92 | 104 | Dose | +2 | +4 | +8 | HV may be turned into in- |
| | Days and Window | | | | | | | | | | | clinic visit per agreement |
| | 379 ± 2 | 393 ± 4 | 421 ± 4 | 477 ± 4 | 561 ± 4 | 645 ± 4 | 729 ± 4 | | ±2 | ±4 | ±4 | between patient and site but |
| | Visit Type | | | | | | | | | | | following the same HV |
| Procedure | HV | X | X | X | X | X | X | X | HV | X | X | assessments. |
| Full PE including | | X | X | | | | | X | | X | | |
| weight | | | | | | | | | | | | |
| Abbreviated PE | | | | X | X | X | | | | | | Abbreviated PE = a |
| | | | | | | | | | | | | targeted PE that |
| | | | | | | | | | | | | includes, at a |
| | | | | | | | | | | | | minimum, body system |
| | | | | | | | | | | | | relevant examination |
| | | | | | | | | | | | | based on Investigator |
| | | | | | | | | | | | | judgement and |
| | | | | | | | | | | | | patient symptoms. |
| Vital signs | | X | X | X | X | X | | X | | X | X | |
| N meningitidis | continuous monitoring | | | | | | | continuous monitoring | | | | |
| vaccination status | | | | | | | | | | | | |
| assessment/ | | | | | | | | | | | | |
| vaccination, if | | | | | | | | | | | | |
| needed | | | | | | | | | | | | |
| Laboratory | X | X | X | X | X | X | X | X | X | X | X | VHS will collect blood |
| assessments | | | | | | | | | | | | samples at HV. |
| (including chemistry, | | | | | | | | | | | | |
| hematology, | | | | | | | | | | | | |
| urinalysis, and | | | | | | | | | | | | |
| LFTs) | | | | | | | | | | | | |
| 12-lead ECG | | | | | | | X | | | | | |
| Drug dispensing | | X | X | X | X | X | X | X | | X | X | At each clinic visit, site |
| | | | | | | | | | | | | should ensure patient has |
| | | | | | | | | | | | | sufficient study drug till their |
| | | | | | | | | | | | | next visit |
| Drug accountability | | X | X | X | X | X | X | X | | X | X | Patients are instructed to |
| | | | | | | | | | | | | bring their study drugs at |
| | | | | | | | | | | | | each clinic visit |
| AE/SAE monitoring | continuous monitoring | | | | | | | continuous monitoring | | | | |
| Concomitant | continuous monitoring | | | | | | | continuous monitoring | | | | |
| medications | | | | | | | | | | | | |
| monitoring | | | | | | | | | | | | |
| Review patient | continuous monitoring | | | | | | | continuous monitoring | | | | Patients to carry card at all |
| safety card | | | | | | | | | | | | times |
| PATIENT REPORTED OUTCOMES | | | | | | | | | | | | All PROs and QoL |
| | | | | | | | | | | | | assessments should be |
| NEI VFQ-25 | | | X | | X | | X | | | | | completed as early as |
| EQ-5D-5L | | | X | | X | | X | | | | | possible during visits, |
| Lawton IADL | | | X | | X | | X | | | | | before ocular assessments |
| OCULAR ASSESSMENTS | | | | | | | | | | | | Ocular assessments are to |
| | | | | | | | | | | | | be performed on both eyes |
| | | | | | | | | | | | | (OU); tests that do not need |
| | | | | | | | | | | | | pupil dilation should be |
| | | | | | | | | | | | | done first |
| BCVA testing on | | | X | X | X | X | X | | | | | Non-dilated, the test will be |
| ETDRS | | | | | | | | | | | | administered first |
| chart′(starting at | | | | | | | | | | | | monocularly (right/left eye) |
| 4 m) | | | | | | | | | | | | and then binocularly (both |
| | | | | | | | | | | | | eyes). |
| LL BCVA testing on | | | | | | | X | | | | | Non-dilated, the test will be |
| ETDRS | | | | | | | | | | | | administered first |
| chart′(starting at | | | | | | | | | | | | monocularly (right/left eye) |
| 4 m) | | | | | | | | | | | | and then binocularly (both |
| | | | | | | | | | | | | eyes). |
| Reading Speed | | | | | | | X | | | | | Non-dilated, the test will be |
| (MNRead or Radner | | | | | | | | | | | | administered first |
| Reading Charts)^{b} | | | | | | | | | | | | monocularly (right/left eye) |
| | | | | | | | | | | | | and then binocularly (both |
| | | | | | | | | | | | | eyes). |
| Mesopic | | | | | | | X | | | | | Non-dilated, only in |
| microperimetry^{c} | | | | | | | | | | | | microperimetry |
| | | | | | | | | | | | | subpopulation |
| Slit lamp | | | X | X | X | X | X | | | | | |
| examination | | | | | | | | | | | | |
| Dilated binocular | | | X | X | X | X | X | | | | | |
| indirect | | | | | | | | | | | | |
| ophthalmoscopy | | | | | | | | | | | | |
| Tonometry/IOP | | | X | X | X | X | X | | | | | Tono-pen or other |
| | | | | | | | | | | | | calibrated tonometers can |
| | | | | | | | | | | | | be used. Goldmann |
| | | | | | | | | | | | | applanation tonometry must |
| | | | | | | | | | | | | also be used to verify the |
| | | | | | | | | | | | | reading of ≥30 mmHg |
| | | | | | | | | | | | | occurring at any time. |
| FAF | | | X | X | X | X | X | | | | | Recommended order of |
| NIR | | | X | X | X | X | X | | | | | imaging is FAF, NIR, |
| SD-OCT | | | X | X | X | X | X | | | | | SD-OCT, fluorescein |
| Fluorescein | | | | | | | X | | | | | angiography, CFP. |
| angiography | | | | | | | | | | | | Administration of artificial |
| CFP | | | | | | | X | | | | | tears between acquisition of |
| | | | | | | | | | | | | each eye for FAF/NIR and |
| | | | | | | | | | | | | SD-OCT is recommended. |

^{a}If optimal dose is identified, all patients who have completed Week 52 will start the transition to the optimal dose at the next scheduled visit.
^{b}Reading speeds will only be assessed if charts are available in the local language.
^{c}Microperimetry should be performed prior to any imaging.
Abbreviations:
AE = adverse event; BCVA = best-corrected visual acuity; CFP = color fundus photography; ECG = electrocardiogram; ETDRS = Early Treatment Diabetic Retinopathy Study; EQ-5D-5L = EuroQol 5-dimension 5-level questionnaire; FAF = fundus autofluorescence; h = hours; HV = home visit; IADL = Instrumental Activities of Daily Living; IOP = intraocular pressure; LFT = liver function test; LL BCVA = low luminance best-corrected visual acuity; MNRead = Minnesota Low-Vision Reading Test; NEI VFQ-25 = National Eye Institute Visual Function Questionnaire, 25-item version; NIR = near infrared reflectance; PE = physical examination; OU = oculus uterque/both eyes; PRO = patient reported outcome; QoL = quality of life; SAE = serious adverse event; SD-OCT = spectral domain optical coherence tomography; VHS = visiting health service

TABLE 10
Schedule of Activities for Early Termination, Taper, and Follow-up

| | | | | | Follow-up | |
|---|---|---|---|---|---|---|
| | Unscheduled | Early | Taper | | (EOS) | |
| | Visit^{a} | Termination | 6 days^{b} | | 30 days | |
| | Visit | | | | | |
| | | | T1 | T2 | | |
| | Weeks | | | | | Notes |
| | Days and Window | | | | | X = in-clinic visit; |
| | | | D 1 to | D 4 to | 30 + 7 post | HV = home visit by VHS; HV may be turned |
| | | | D 3 | D 6 | last dose | into in-clinic visit per agreement between |
| | Visit Type | | | | | patient and site but following the same HV |
| | X | X | HV | HV | X | assessments. |
| Full physical examination | X | X | | | X | |
| including weight | | | | | | |
| Abbreviated PE | | | | | | Abbreviated PE = a targeted PE that |
| | | | | | | includes, at a minimum, body system |
| | | | | | | relevant examination based on Investigator |
| | | | | | | judgement and patient symptoms |
| Vital signs | X | X | | | X | |
| N meningitidis | X | X | X | X | | |
| vaccination status | | | | | | |
| assessment/ vaccination, | | | | | | |
| if needed | | | | | | |
| Laboratory assessments | X | X | | | X | VHS will collect blood samples at HV. |
| (including chemistry, | | | | | | |
| hematology, urinalysis, | | | | | | |
| and LFTs) | | | | | | |
| PD (Bb and AP activity) | | X | | | | ET samples for PD and biomarkers only |
| Biomarkers | | X | | | | applicable for the first 52 weeks. No |
| | | | | | | samples after Week 52. |
| 12-lead ECG | | X | | | X | Single tracing. |
| Drug dispensing | | X | ^{Xb} | X^{b} | | Patients are provided with sufficient study |
| | | | | | | drug to last until their next visit. |
| Drug accountability | | X | X | X | X | Patients are instructed to bring their study |
| | | | | | | drugs at each visit. |
| AE/SAE review | X | X | X | X | X | |
| Concomitant medications | X | X | X | X | X | |
| review | | | | | | |
| Review patient safety | X | X | X | X | X | Patients to carry card at all times. |
| card | | | | | | |
| PATIENT REPORTED OUTCOMES | | | | | | All PROs and QoL assessments should be |
| | | | | | | completed as early as possible during visits. |
| NEI VFQ-25 | | X | | | | |
| EQ-5D-5L | | X | | | | |
| Lawton IADL | | X | | | | |
| OCULAR ASSESSMENTS | | | | | | Ocular assessments are to be performed on |
| | | | | | | both eyes (OU); tests that do not need pupil |
| | | | | | | dilation should be done first. |
| BCVA testing on ETDRS | X | X | | | | Non-dilated, the test will be administered |
| chart (starting at 4 m) | | | | | | first monocularly (right/left eye) and then |
| | | | | | | binocularly (both eyes). |
| LL BCVA testing on | | X | | | | Non-dilated, the test will be administered |
| ETDRS chart (starting at | | | | | | first monocularly (right/left eye) and then |
| 4 m) | | | | | | binocularly (both eyes). |
| Reading Speed | | X | | | | Non-dilated, the test will be administered |
| (MNRead or Radner | | | | | | first monocularly (right/left eye) and then |
| Reading Charts)^{c} | | | | | | binocularly (both eyes). |
| Mesopic microperimetry^{d} | | X | | | | Non-dilated; only in microperimetry |
| | | | | | | subpopulation. |
| Slit lamp examination | X | X | | | | |
| Dilated binocular indirect | X | X | | | | |
| ophthalmoscopy | | | | | | |
| Tonometry/IOP | X | X | | | | Tono-pen or other calibrated tonometers |
| | | | | | | can be used. Goldmann applanation |
| | | | | | | tonometry must also be used to verify the |
| | | | | | | reading of ≥30 mmHg occurring at any time. |
| FAF | X | X | | | | Recommended order of imaging is FAF, |
| NIR | X | X | | | | NIR, SD-OCT, fluorescein angiography, |
| SD-OCT | X | X | | | | CFP. Administration of artificial tears |
| Fluorescein angiography | | X | | | | between acquisition of each eye for |
| CFP | X | X | | | | FAF/NIR and SD-OCT is recommended. |

^{a}Unscheduled visits for safety and worsening of vision; additional assessments may be performed if deemed necessary by the Investigator
^{b}Tapering will be masked; masked blister packs will be provided to patients possibly to home, at ET, or unscheduled visit, etc. See Table 14.
^{c}Reading speeds will only be assessed if charts are available in the local language.
^{d}Microperimetry should be performed prior to any imaging.
Abbreviations:
AE = adverse event; AP = alternative pathway; Bb = Bb fragment of complement factor B; BCVA = best-corrected visual acuity; CFP = color fundus photography; D = Day; ECG = electrocardiogram; ETDRS = Early Treatment Diabetic Retinopathy Study; EOS = end of study; EQ-5D-5L = EuroQol 5-dimension 5-level questionnaire; ET = early termination; FAF = fundus autofluorescence; IADL = Instrumental Activities of Daily Living; IOP = intraocular pressure; LFT = liver function test; LL BCVA = low luminance best-corrected visual acuity; MNRead = Minnesota Low-Vision Reading Test; NEI VFQ-25 = National Eye Institute Visual Function Questionnaire, 25-item version; NIR = near infrared reflectance; OU = oculus uterque/both eyes; PE = physical examination; QoL = quality of life; SAE = serious adverse event; SD-OCT = spectral domain optical coherence tomography; VHS = visiting health service

### Outcome Measures

### Primary Outcomes: Square Root of GA Lesion Area

The primary efficacy outcome measure of this study is the change in the square root (sqrt) of total GA lesion area (in mm) from Baseline at Week 52 as assessed by fundus autofluorescence (FAF) in the study eye. Although the kinetics of GA progression are highly variable among individual patients, a growing body of evidence suggests that specific characteristics of the lesions may be important in predicting disease progression and outcomes. Therefore, the quantitative measurement of total GA lesion area progression by FAF imaging has become an acceptable anatomic marker used as clinical endpoint for clinical studies (see Holz et al. (2007), supra; Holz et al. (2018), supra; Liao et al. (2020), supra; and Sadda et al. (2016), supra). The sqrt transformation of the GA lesion area measurements eliminates variability dependence on baseline lesion size (see Feuer et al. (2013), supra; and Yehoshua et al. (2014), supra).

### Secondary Outcomes: Anatomical and Functional Outcomes

A combined anatomical (with multimodal imaging techniques) and corresponding functional assessment strategy that incorporates many different aspects of visual function is appropriate in capturing changes in visual function associated with progressive GA changes from AMD.

In this study, a combination of anatomical and functional tests to assess disease progression and visual deficits will be used as outcome measures.

Anatomical outcomes will be based on changes in GA lesion area using different imaging modalities.

Functional outcomes will be based on changes in best-corrected visual acuity (BCVA) scores, low luminance visual acuity (LLVA) scores, the low luminance deficit (LLD) calculated from these 2 scores (BCVA-LLVA), and reading speeds.

Patient-reported outcomes (PRO) will be assessed using the National Eye Institute Visual Function Questionnaire, 25-item version (NEI VFQ-25).

### Exploratory Analyses

Exploratory analyses will be performed based on anatomical and functional outcomes.

Disease progression of AMD will be followed by quantifying conversion from one disease stage to another based on disease classification described in Table 1.

Functional outcomes will be based on changes in BCVA scores, LLVA scores, the LLD calculated from these 2 scores in fellow eye and both eyes. Functional retinal response including number of scotomatous points and change in macular sensitivity will be assessed by mesopic microperimetry in the microperimetry eligible subpopulation.

Anatomical outcomes will be based on anatomical measures on the fellow eye and both eyes combined, including changes in the area and sqrt area of the total GA lesion, changes in drusen volume, and incidence of patients with conversion from incomplete retinal pigment epithelium and outer retinal atrophy (iRORA) to complete retinal pigment epithelium and outer retinal atrophy (cRORA), as measured by spectral-domain optical coherence tomography (SD-OCT).

### Patient Reported Outcomes

Both nAMD and GA can lead to the loss of vision that may be severe and irreversible, resulting in a significant loss of QoL and inability to perform activities of daily living. Collection of patient reported outcomes using quality-of-life instruments and visual functioning questionnaires in clinical trials has been recommended by regulatory bodies (see Csaky et al. (2017), supra). Change in QoL and daily living functioning are measured as exploratory endpoints using validated instruments.

### Patient Population

Epidemiology studies show a substantial increase in AMD risk with age. Prevalence is highest in age groups ≥75 years (see Augood et al., Arch Ophthalmol. 2006; 124(4):529-535; and Lambert et al., Prog Retin Eye Res. 2016; 54:64-102).

The study population of >60 years of age assures a balanced representation of the GA patient population with a reported mean (±standard deviation [SD]) age of 79±8 years (see Yaspan et al., Sci Transl Med. 2017; 9(395)). This also provides the opportunity to capture the photoreceptor degeneration and conversion events and rules out unilateral GA in younger patients that may be due to other diseases such as myopia, uveitis, post trauma, or radiation.

### Justification for Dose

One of the key objectives for this study is to test potentially efficacious and safe doses and dose regimens of danicopan and to support the selection of an appropriate dose for subsequent clinical development.

The 100 mg BID, 200 mg BID, and 400 mg QD dose regimens proposed for this study are based on simulation results. Plasma PK exposures for these proposed doses are predicted to remain below the identified exposures where tolerability limit was observed in a MAD study. These doses are predicted to achieve >90% alternative pathway hemolysis (APH) inhibition for the entire dose interval in the potential eye tissue targets: the retina and the choroid RPE retinal side (melanosomes), and partial-to-complete APH inhibition in the choroid RPE capillary bed side (peripheral). Based on data from the literature, retina and choroid RPE on the retinal side are more likely to be the target eye tissues. However, the choroid RPE on the capillary bed side cannot be completely ruled out as a potential target. The proposed doses allow for the assessment of two different dosing intervals, i.e., BID vs QD. The 400 mg QD dosing regimen, if shown to be safe and effective, would be more convenient for patients and encourage better compliance relative to bid dosing. An up to 2-fold uncertainty of model prediction on PK and APH inhibition in the target eye tissues is anticipated as the physiological eye parameters in humans were translated from Dutch-Belted rabbits.

### Definitions of Study Periods and End of Study

Primary Evaluation Period: Day 1 to Week 52 (Table 8).

Secondary Evaluation Period: starts from Week 52 and ends at Week 104 (Table 9). The primary and secondary periods are masked.

Study completion: a patient is considered to have completed the study if he/she has completed all periods of the study, including the OLE Period and the last scheduled procedure shown in the SoA.

Early termination (ET) or discontinuation: a patient is considered to early terminate from the study if the patient is discontinued from the study before the last visit as described in Table 3.

Follow-up: a follow-up visit is scheduled 30 (+7) days after the last dose (including taper doses, Table 10) of study drug.

End of study (EOS): The end of the study is defined as the date the last patient completes the last visit (including the Taper and Follow up; Table 10).

### Study Population

### Inclusion Criteria

### General Inclusion Criteria

1. Age ≥60 years, male or female.
2. All patients must be vaccinated against meningococcal infections within 3 years prior to, or at the time of, initiating study drug. Patients who initiate study drug treatment less than 2 weeks after receiving a meningococcal vaccine must receive treatment with appropriate prophylactic antibiotics until 2 weeks after vaccination.
3. For female patients, confirmation of non-childbearing potential based on follicle stimulating hormone (FSH) test at Screening only.
4. For nonsterile male patients, agreement to use a highly effective or acceptable method of contraception with their partner(s) of childbearing potential from the first day of dosing to 90 days after their last dose of study drug. Males who are surgically sterile do not need to employ additional contraception.
5. For male patients, agreement not to donate sperm while enrolled in this study and for 90 days after their last dose of study drug.
6. Capable of giving signed informed consent, which includes compliance with the requirements and restrictions listed in the informed consent form (ICF) and in this protocol.

### Ocular Inclusion Criteria

At Screening, the eye that meets all eligibility criteria is designated as the study eye and the other eye will be the fellow eye. In case of bilateral GA wherein both eyes are eligible, the right eye is designated as the study eye. In cases where both eyes are eligible, if eligibility status of the designated study eye changes during review of eligibility criteria on Day 1 prior to randomization, the other eye can be designated as the study eye if all eligibility criteria are met.

To be eligible, there should be a presentation of GA secondary to AMD in at least one eye, characterized by:
1. The study eye must have the specified VA (range of 84 to 24 letters; 20/20 to 20/320) using Early Treatment Diabetic Retinopathy Study (ETDRS) charts at starting distance of 4 meters.
2. Adequate clarity of ocular media, adequate pupillary dilation, and fixation assessed by slit lamp examination and indirect ophthalmoscopy to permit the collection of good quality images as determined by the Investigator.
3. Axial length ≤26.0 mm measured by biometry (if available) and spherical equivalent refractive error ≤6.0 diopter of myopia.
   For patients with history of refractive or cataract surgery in the study eye, axial length ≤26.0 mm measured by biometry (if available) OR preoperative spherical equivalent refractive error ≤6.0 diopter of myopia.
4. The entire GA lesion must be completely visualized on the macula centered field 2 of the FAF image, must be able to be imaged in its entirety, and must not be contiguous with any areas of peripapillary atrophy.
5. Total GA lesion area of 0.5 to 17.76 mm² (˜0.2 to 7 disc area [DA]) per eye measured by FAF. If GA is multifocal, at least one focal lesion must be ≥0.5 mm² (˜0.2 DA).
6. The entire GA lesion must be >1 μm outside of the foveal center.

### Exclusion Criteria

### Ocular Exclusion Criteria

1. GA in the study eye due to cause other than AMD (pathological myopia, monogenetic macular dystrophies, e.g., Stargardt/cone-rod dystrophy) or toxic maculopathies (e.g., chloroquine/hydroxychloroquine maculopathy) per Investigator's judgement.
2. GA and concomitant nAMD in the study eye.
3. Have previously received intravitreal anti vascular endothelial growth factor (VEGF) injections in study eye for intraocular vascular disease.
4. Have previously received any stem cell or gene therapy for any ophthalmological condition in either eye.
5. Previous participation in interventional clinical studies for any ophthalmic indications in the study eye regardless of route of administration within the last 3 months or 5 half-lives of the investigational product (whichever is longer).
6. Previous laser photocoagulation for nAMD, diabetic macular edema, retinal vein occlusion and proliferative diabetic retinopathy in the study eye.
7. Previous photodynamic therapy (visudyne) or transpupillary thermotherapy in the study eye.
8. Previous external beam radiation therapy and/or any other irradiation (e.g., isotope, charged particle, photon, x-ray) to the study eye and respective orbit, head, and/or neck.
9. Previous intravitreal delivery of steroid or device implantation, with the exception of intraocular lens, in the study eye. A single intravitreal steroid injection for cystoid macular edema after cataract surgery ≥3 months prior to Screening is permitted in the study eye.
10. Presence of an active ocular diseases in the study eye that in the opinion of the Investigator compromises or confounds visual function or interferes with study assessments, including but not limited to cataract, uveitis, keratitis, scleritis or endophthalmitis, vitreous hemorrhage, other macular diseases (e.g., clinically significant epiretinal membrane, full thickness macular hole, RPE tear in macula), central serous retinopathy, uncontrolled glaucoma, proliferative diabetic retinopathy.
11. History of any of the following in the study eye:
   a. retinal detachment or macular hole
   b. glaucoma-filtering surgery including microinvasive glaucoma surgery (MIGS)
   c. vitrectomy, submacular surgery, or any surgical intervention for AMD
   d. prophylactic subthreshold laser treatment for AMD
   e. intraocular surgery (including lens replacement surgery) within 3 months prior to randomization.
12. History of any recurrent infectious or inflammatory eye disease in either eye.
13. If the following changes occur between Screening and Day 1 in the study eye, as determined by eligibility review before randomization at Day 1:
   a. the Snellen equivalent is no longer between 20/20 to 20/320 OR
   b. significant anatomical changes (i.e., large subretinal hemorrhage, RPE rip, pigment epithelial detachment, or other conditions that meet the exclusion criteria per Investigator's discretion).

### General Exclusion Criteria

1. Known or suspected complement deficiency.
2. History of N meningitidis infection.
3. Active bacterial or viral infection, a body temperature >38° C. (100.4ºF) on 2 consecutive daily measures, evidence of other infection, or history of any febrile illness within 14 days prior to first study drug administration.
4. History of malignant disease within the past 5 years or ongoing, including solid tumors and hematologic malignancies (with the exception of basal cell and squamous cell carcinomas of the skin that have been completely excised and are considered cured).
5. Abnormal liver function tests, defined as:
   a. ALT, aspartate aminotransferase (AST), alkaline phosphatase (ALP) or direct bilirubin >2× upper limit of normal (ULN) at Screening
   b. Patients with Gilbert's Syndrome will not be excluded. If increased bilirubin is suggestive of Gilbert's syndrome, the patient has to provide documentation of the diagnosis or will be tested for this condition.
6. Evidence of hepatitis B infection (positive hepatitis B surface antigen [HbsAg] or positive core antibody (anti-HBc) with negative surface antibody [anti-HBs]) or hepatitis C viral infection (HCV antibody positive), except for patients with documented successful treatment and documented successful treatment and documented sustained virologic response (SVR) at Screening.
7. Evidence of human immunodeficiency virus (HIV antibody positive) infection at Screening.
8. Estimated glomerular filtration rate <30 mL/min/1.73 m² and/or are on dialysis. eGFR will be calculated using the Chronic Kidney Disease Epidemiology Collaboration (CKD EPI) equation.
9. Hypersensitivity to the investigational drug (danicopan) or any of its excipients, or to fluorescein sodium for injection (known fluorescein hypersensitivity previously successfully pre-medicated can be performed per Investigator discretion).
10. Participation in non-ophthalmologic clinical trials involving administration of another investigational medicinal product within 5 half-lives (if known) or within 30 days for systemic non-biologics or 6 months for biologics, whichever is longer.
11. History or presence of any medical or psychological condition that, in the opinion of the Principal Investigator, would make the patient inappropriate for the study or unable to comply with study procedures or put the patient at undue risk or confound the results of the study.

### Microperimetry Eligibility Criteria

A subset of patients will be included in a microperimetry subpopulation. The eligibility criteria for microperimetry are as follows:
Meeting all the inclusion criteria and none of the exclusion criteria listed above.
Eyes able to detect fixation target.
Total elapsed time to complete a microperimetry test is ≤30 minutes in duration per test.
Fixation losses must be ≤20%.

### Lifestyle Considerations

### Meals and Dietary Restrictions

Danicopan is taken orally with food. See below for details of study drug administration.

### Screen Failures

Screen failures are defined as patients who consent to participate in the clinical study but are not subsequently randomly assigned to study drug. A minimal set of screen failure information is required to ensure transparent reporting of screen failure patients to meet the Consolidated Standards of Reporting Trials publishing requirements and to respond to queries from regulatory authorities. Minimal information includes demography, screen failure details (e.g., failed eligibility criteria), and any Aes, including any serious adverse events (SAEs) and any related concomitant medication, occurring during the Screening Period.

Individuals who do not meet the criteria for participation in this study (screen failure) due to a reason that is expected to resolve or has resolved may be rescreened based on discussion and agreement between the Investigator and the Medical Monitor. Participants who are rescreened outside of the Screening window are required to sign a new ICF. During rescreening, the patient must repeat all required screening assessments as defined in Screening visit in the SOA.

### Study Intervention

For this study, the intervention is the administration of study drugs shown in Table 11.

TABLE 11
Study Drugs

| | Danicopan | Placebo |
|---|---|---|
| Type | Small molecule drug | Placebo |
| Dose formulation | tablet | tablet |
| Unit dose strength | 100 mg | N/A |
| Dose levels | 100 mg BID, 200 mg | matching placebo |
| | BID, 400 mg QD | |
| Route of | oral | oral |
| administration | | |
| Use | Experimental | Placebo control |
| IMP and NIMP | IMP | IMP |
| Sourcing | provided by Sponsor | provided by Sponsor |
| Packaging and | Tablets are provided in | Tablets are provided in |
| labeling | blister packs and bottles | blister packs and bottles |
| Other names | ALXN2040, ACH-0144471 | N/A |
| and aliases | | |

Abbreviations: BID = twice daily; IMP = investigational medicinal product; N/A = not applicable; NIMP = non-investigational medicinal product; QD = once daily

### Study Drug Administration

The study drugs will be dispensed to the patients at the clinic visits, mostly to be taken at home at the specified dosing regimen. At each clinic visit, the site should ensure that the patient has sufficient drug supply until the next clinic visit. Patients will be instructed to bring back all unused tablets at each clinic visit for accountability.

At certain clinic visits that require PK sample collection, patients should be instructed to administer their study drugs at the clinic for predose blood sample collection (Table 12).

TABLE 12
Pharmacokinetic and Pharmacodynamic Blood Sampling During
the Primary Evaluation Period (Up to Week 52)

| | BL | | Week 4 | | | Week 16, 28, 36, 52 | | |
|---|---|---|---|---|---|---|---|---|
| | Time After Dosing (Hour ±10 min) | | | | | | | |
| | Predose² | 2 h | Predose² | 2 h | 4 h | Predose² | 2 h | ET |
| PK plasma samples¹ | X | X | X | X | X | X | X | |
| PD: AP activity³ | X | X | X | X | X | X | X | X |
| PD: Bb concentration³ | X | | X | | | X | | X |
| Plasma/serum samples for | X | | X | | | X | | X |
| additional non-genetic biomarker | | | | | | | | |
| testing³ | | | | | | | | |

¹PK samples will be drawn predose and postdose.
²Predose samples: Patients should be instructed to take their dose onsite, with a snack or meal. The time when the previous dose was taken (if possible, 12 hours prior to blood draw) should be recorded.
³APW samples will be collected at the same time as the PK samples and once at ET. Bb and biomarkers will be collected predose and also once at ET.
Abbreviations:
AP = alternative pathway; APW = alternative pathway Wieslab; Bb = Bb fragment of complement factor B; BL = Baseline; ET = early termination; h = hour; min = minutes; PD = pharmacodynamic(s); PK = pharmacokinetic(s)

The study drugs (danicopan or matching placebo) will be provided as tablets which are identical in appearance. The composition of the masked study treatments is shown in. The composition of the masked study treatments is shown in Table 13.

TABLE 13
Study Drug Administration (Weeks 1 to 104)

| | Dose Group* | | | |
|---|---|---|---|---|
| Timing | 100 mg BID | 200 mg BID | 400 mg QD | Placebo |
| Morning dose, with | 100 mg | 100 mg | 100 mg | placebo |
| food and water | placebo | 100 mg | 100 mg | placebo |
| | placebo | placebo | 100 mg | placebo |
| | placebo | placebo | 100 mg | placebo |

Approximately 12 hours

| | | | | |
|---|---|---|---|---|
| Evening dose, with | 100 mg | 100 mg | placebo | placebo |
| food and water | placebo | 100 mg | placebo | placebo |

*Doses are given as tablets in 100 mg strengths
Abbreviations: BID = twice daily; QD = once daily

At home, patients will take their tablets twice daily with food and water, one dose of 4 tablets in the morning, and a second dose of 2 tablets in the evening. The time interval between the 2 doses should be approximately 12 hours.

At certain clinic visits (see SoA), patients will be instructed to take their morning dose of 4 tablets at the clinic to enable predose blood collection.

### Open-Label Extension Period

During the open-label period, danicopan tablets will be provided in bottles and taken at the selected optimal dose, with food and water.

### Dose Tapering

In a MAD Study, ALT elevations were observed at high doses in 2 subjects after dosing was ceased. The elevations were transient and not considered of clinical significance. However, this temporal relationship suggests that the sudden withdrawal of FD inhibition may be associated with liver enzyme elevations. A dose taper was instituted in subsequent studies as a risk mitigation measure.

If possible, tapering should be initiated during dose interruptions, study discontinuation, or study completion. The dose of danicopan or placebo will be tapered over a 6-day period according to the dose tapering regimen described in Table 14. In case of discontinuation, the patient will complete the ET visit, if possible, prior to tapering and should take the study drug per protocol until the Tapering period begins.

Tapering doses will be taken only in the morning with food and water. Masking must be maintained during tapering occurring within the Masked Treatment Period.

TABLE 14
Study Drug Taper Schedule

| | Dose Group | | | | | | | |
|---|---|---|---|---|---|---|---|---|
| | 100 mg BID | | 200 mg BID | | 400 mg QD | | Placebo | |
| | Taper | Masked | Taper | Masked | Taper | Masked | Taper | Masked |
| Timing | dose | dose | dose | dose | dose | dose | dose | dose |
| Taper Period 1 | 100 mg | 100 mg | 200 mg | 100 mg | 200 mg | 100 mg | placebo | placebo |
| (Taper Days 1 to | QD | placebo | QD | 100 mg | QD | 100 mg | | placebo |
| 3), mornings^{a} | | | | | | | | |
| Taper Period 2 | 0 mg | placebo | 100 mg | 100 mg | 100 mg | 100 mg | placebo | placebo |
| (Taper Days 4 to | | | QD | | QD | | | |
| 6), mornings^{a} | | | | | | | | |

Error! Reference source not found. During taper, patients will take tablets in the morning with food and water.
Abbreviations:
bid = twice daily; qd = once daily
T1 and T2 visits are done on Day 3 and Day 6, respectively, by visiting healthcare service.
T1 should assess safety and give instructions to taper dosing. T2 should give instructions to terminate dosing.

In case the tapering is not tolerated, there should be a discussion between the Investigator and the Medical Monitor.

At the end of the Tapering Period, the patient will be scheduled for a Follow up visit 30 (+7) days after the last taper dose of the study drug.

Data collected at the time of study drug discontinuation, Taper, and Follow up and for any further evaluations that need to be completed are provided in the SoA (Tables 8-10).

### Vaccines

To reduce the risk of meningococcal infection, all patients must be vaccinated against meningococcal infections within 3 years prior to, or at the time of, initiating study drug. Vaccines against serotypes A, C, Y, W135, and B where available, are recommended to prevent common pathogenic meningococcal serotypes. Patients who initiate study drug treatment less than 2 weeks after receiving a meningococcal vaccine must receive treatment with appropriate prophylactic antibiotics from the first day of study drug treatment until 2 weeks after vaccination.

Patients must be vaccinated or revaccinated according to current national vaccination guidelines or local practice for vaccination use with complement inhibitors. Vaccination may not be sufficient to prevent meningococcal infection. All patients should be monitored for early signs of meningococcal infection, evaluated immediately if infection is suspected, and treated with appropriate antibiotics, if necessary.

Patients should be vaccinated or revaccinated against other pathogens according to current national vaccination guidelines or local practice for vaccination use as part of standard of care of underlying disease and age group.

### Switching to Optimal Dose

If an optimal dose is identified, all patients with at least 52 weeks of treatment, as specified in the SoA will transition to the optimal dose at the next scheduled visit when drug supply is made available. Placebo patients will be rerandomized to one of the 3 active treatment groups at Week 52, or switched to the optimal dose, if already identified. Masking will be maintained till the end of the study.

The timing of transitioning to optimal dose will be programmed into the IVRS/IWRS to ensure that masking is maintained till then end of the study.

### Missed Doses

The study drug (danicopan or placebo) should be taken as prescribed by the protocol and without interruption during the course of the study, whenever possible. In case of a dose missed (defined as dose at a single timepoint, morning or evening) inadvertently, the patient should continue with administration of a regularly scheduled next dose. Adding the missed dose at the next dosing time is not allowed. Information on missed doses should be recorded in the eCRF.

### Dose Interruptions

If the study drug (danicopan or placebo) has to be stopped or interrupted for any reason, assessment should be made if the stop is temporary and discussion about the reasons for interruption and plans for potential re-initiation should occur between the Investigator and the Medical Monitor. Re-initiation of treatment with the study drug should be done under careful clinical monitoring, including laboratory monitoring and after consultation with the Medical Monitor. If upon resumption of treatment, the patient has a recurrence of the event, permanent discontinuation should be considered.

Any temporary preplanned treatment interruption should be discussed between the Investigator and the Medical Monitor and tapering should be considered (refer to Table 14).

The patient should continue participation until the end of the study unless the pre-specified events for treatment discontinuation have been met. Any interruption of study drug and the reason for the interruption should be fully documented in the source documents and eCRF.

### Permanent Dose Discontinuation

If the study drug (danicopan or placebo) is permanently discontinued for any reason, the dose of danicopan or placebo will be tapered over a 6-day period as described in Table 14.

A patient who permanently discontinues the study drug is also permanently discontinued from the study.

### Study Assessment and Procedures

Study procedures and their timing are summarized in the SoA (Tables 8-10). Protocol waivers or exemptions are not allowed.

There are two types of study visits: in-clinic visits, home visits by a visiting health service (in countries where available). All visits must be complied with according to the SoA. Patients may opt to convert home visits to in-clinic visits per agreement with site personnel, but the home visit assessments will still be followed. Changes in visit type should be noted in source documentation.

Immediate safety concerns should be discussed with Alexion immediately upon occurrence or awareness to determine if the patient should continue or discontinue study drug.

Adherence to the study design requirements, including those specified in the SoA, is essential and required for study conduct.

Procedures conducted as part of the patient's routine clinical management (e.g., blood count) and obtained before signing of the ICF may be utilized for screening or baseline purposes provided the procedures met the protocol specified criteria and were performed within the time frame defined in the SoA (Tables 8-10). The list of clinical laboratory tests is provided in Table 15.

TABLE 15
Protocol-Required Laboratory Assessments

| Hematology | Chemistry | Urinalysis | Other Assessments¹ |
|---|---|---|---|
| Complete blood count | Liver function tests: | Analysis including: | Biomarkers: |
| (CBC), including: | Alanine | Bilirubin | Alternative pathway |
| Red blood cell (RBC) | aminotransferase (ALT) | Color | activity |
| count | Alkaline phosphatase | Glucose | Bb |
| White blood cell (WBC) | Aspartate | Ketones | C3 |
| count | aminotransferase (AST) | Leukocytes | Classical pathway |
| WBC differential | Gamma-glutamyl | Nitrite | activity |
| (absolute and | transferase (GGT) | Occult blood | Factor D |
| percent): | Albumin | pH | Serology : |
| neutrophils | Bicarbonate (HCO₃) | Protein | HCV Ab |
| lymphocytes | Bilirubin (fractionated)² | Specific gravity | HbsAg |
| monocytes | Blood urea nitrogen | Urobilinogen | HbcAg |
| eosinophils | (BUN) | Microscopic | HIV Ab |
| basophils | Calcium | examination of | UGT1A1 (Gilbert's)⁶ |
| Hematocrit (Hct) | Calculated eGFR³ | sediment | Genetic biomarkers |
| Hemoglobin (Hgb) | Chloride | | (optional) |
| Mean corpuscular | C-reactive protein | | Drug concentrations for |
| volume (MCV) | (CRP) | | PK |
| Mean corpuscular | Creatine kinase⁴ | | FSH for females |
| hemoglobin (MCH) | Creatinine | | |
| Mean corpuscular | Glucose⁵ | | |
| hemoglobin | HbA1c (glycated | | |
| concentration (MCHC) | hemoglobin) | | |
| Mean platelet volume | Lactate dehydrogenase | | |
| (MPV) | Lipid profile | | |
| Platelet count | including: | | |
| PT/PTT/INR | Cholesterol/HDL ratio | | |
| Red cell distribution | High-density lipoprotein | | |
| width (RDW) | cholesterol (HDL-C) | | |
| Reticulocyte count | Low-density lipoprotein | | |
| (absolute and percent) | cholesterol (LDL-C) | | |
| | Non-HDL-C | | |
| | Total cholesterol | | |
| | Triglycerides | | |
| | Very low-density | | |
| | lipoprotein cholesterol | | |
| | (VLDL-C) | | |
| | Potassium | | |
| | Sodium | | |
| | Total protein | | |
| | Uric acid | | |

¹Check the SoA for specific time points when these tests should be done.
²Fractionate and obtain measurements of direct and indirect bilirubin for all patients. If indirect bilirubin levels are >ULN at Screening but ALT and AST are normal, test for Gilbert's syndrome.
³Provide estimated glomerular filtration rate (eGFR) based on Chronic Kidney Disease-Epidemiology Collaboration (CKD-EPI) creatinine equation (2009) for patients ≥19 years of age and based on the “bedside Schwartz” equation (2009) for patients <19 years of age.
⁴Perform at Baseline, and then subsequently only as a reflex if AST > the upper limit of normal.
⁵If glucose is >ULN, reflexively test HbA1c.
⁶Test only if medical or family history suggestive of Gilbert's Syndrome.

### Screening

During Screening, there should be an ongoing discussion between the Investigator and the Medical Monitor regarding eligibility criteria.

The Principal Investigator or designee is responsible for administering and obtaining freely given informed consent before the patient enters the study and before any study related procedures are performed. Each patient will sign (written or electronic) an ICF. This may include additional consent forms for HIV testing or other procedures which may be performed prior to patients being accepted into the study.

All screening evaluations (SoA, Table 8) must be completed during the Screening Period and then reviewed at Day 1 to confirm that potential patients meet all eligibility criteria. The Investigator will maintain a screening log to record details of all patients screened and to confirm eligibility or record reasons for screening failure, as applicable.

Individuals who do not meet the criteria for participation in this study (screen failure) due to a reason that is expected to resolve or has resolved may be rescreened based on discussion and agreement between the Investigator and the Medical Monitor. Participants who are rescreened outside of the Screening window are required to sign a new ICF. During rescreening, the patient must repeat all required screening assessments as defined in Screening visit in the SoA.

### General Screening

As part of the screening process, patients will be evaluated for vaccination requirements as detailed herein. A Patient Safety Card will be issued at Screening.

A window of up to 6 weeks is permitted to allow screening and any required vaccinations. Screening procedures may be spread over more than 1 visit within the 6-week Screening Period. The screening clinic and laboratory procedures listed in Table 15 must be performed and documented prior to dosing. This will include a review of the inclusion and exclusion criteria. The patient's medical history will be reviewed, and a complete physical examination will be conducted.

Female participants in this study are postmenopausal and therefore not of child-bearing potential. An FSH test will be performed at Screening for confirmation.

If screening laboratory assessments show elevated indirect bilirubin levels in conjunction with normal liver function tests (AST and ALT), or if the patient has a history of unexplained jaundice, unexplained high bilirubin levels, or a history otherwise suggestive of Gilbert's syndrome, the patient will be tested for this condition. If the patient has a history of Gilbert's syndrome it should be documented as the patient's medical history. Refer to the Laboratory Manual for testing procedure.

### Ocular Screening

At Screening, all patients will undergo a monocular test on both eyes. The “study eye” is defined as the eye that meets all eligibility criteria at Screening. For patients with bilateral GA with both eyes meeting the eligibility criteria, the right eye will be taken as the study eye. The other eye will be used as the “fellow eye.”

The following ocular assessments will be conducted for both eyes during Screening. It is recommended to follow the order of the procedures as listed below. Assessments that require non-dilation of the pupils must be performed first:
BCVA assessed on ETDRS chart at a starting distance of 4 m (perform prior to dilating eyes). The test will be administered first monocularly (right/left eye) and then binocularly (both eyes).
For the microperimetry subpopulation: mesopic microperimetry will be performed on both eyes without dilation. Patient is allowed up to 3 attempts to meet screening criteria.
Slit-lamp examination on both eyes.
Intraocular pressure (IOP) measurement of both eyes (perform prior to dilating eyes; Goldmann applanation tonometry must be used at screening).
Dilated binocular indirect high-magnification ophthalmoscopy on both eyes.
Retinal imaging to be performed in the following recommended order: FAF, near infrared reflectance (NIR), SD-OCT, fluorescein angiography (FA), and color fundus photography (CFP) will be performed per Central Reading Manual and training materials. Administration of artificial tears between acquisition of each eye for FAF/NIR and SD-OCT is recommended. Before any study images and microperimetry are obtained, site personnel, test images, and systems and software (where applicable) will be certified/validated by the reading center as specified in the Central Reading Manual. During the eligibility assessment, if the FAF images were reviewed to be ungradable per Central Reading Center, the FAF images will be repeated as soon as feasible
If available, biometry (preferred) on both eyes to measure axial length. If biometry is not available, spherical equivalent refractive error is to be used.

All ocular images at Screening will be read centrally by trained graders according to a Central Reading Manual. Eligibility assessment by the Central Reading Center based on reading of screening images should be considered by the Investigator when confirming that potential patients meet all eligibility criteria. Discrepancies in eligibility confirmation may be discussed between the Investigator, Medical Monitor, and Central Reading Center.

Prior to randomization at Day 1, the inclusion and exclusion criteria will be reviewed to confirm eligibility. If eligibility status of the designated study eye changes during review of eligibility criteria on Day 1 prior to randomization, the other eye can be designated as the study eye if all eligibility criteria are met.

### Efficacy Assessments

Efficacy will be based on anatomical, functional and health-related QoL assessments at in clinic visits. The assessments should be done in the sequence shown and detailed in the SoA (Tables 8-10):
Patient-reported outcomes
BCVA testing (starting at 4 m)
Low luminance best corrected visual acuity (LL BCVA) testing (starting at 4 m)
Reading Speed (Minnesota Low-Vision Reading Test [MNRead] or Radner Reading Charts)
Mesopic microperimetry for microperimetry subpopulation
Slit lamp examination
Tonometry/IOP
Dilated binocular indirect ophthalmoscopy
Ocular imaging, recommended to be done in the following order: FAF, NIR, SD-OCT, FA, and CFP. Administration of artificial tears between acquisition of each eye for FAF/NIR and SD OCT is recommended.

All ocular images at Screening will be read centrally by trained graders according to a Central Reading Manual.

### Primary Efficacy Assessment

The primary objective of this study is to evaluate the effect of different dosage regimens of danicopan on the progression of GA secondary to AMD.

GA lesion area in mm² will be measured by FAF in the study eye at the time points indicated in Tables 8-10. The total GA lesion area (mm²) will be transformed into sqrt (mm).

FAF provides high-contrast retinal images particularly valuable for the detection of atrophic areas. FAF images will be taken according to the SoA (Tables 8-10) and sent to reading center for total GA lesion area measurement. Analysis of FAF images for the primary efficacy assessment will be centrally performed by trained masked graders per the Central Reading Manual.

### Secondary Efficacy Assessments

Several anatomical and functional measures will be utilized to evaluate the effect of danicopan on GA progression in the study eye. In all cases, assessments that require non-dilated eyes should be performed first prior to dilation.

### Anatomical Measures

GA lesion area in mm² will be measured by FAF for both eyes at the time points indicated in Tables 8-10. The total lesion area will be transformed into sqrt.

### Functional Measures

Monocular BCVA scores, and LLVA scores, and LLD score (BCVA-LLVA) will be assessed by ETDRS chart at a starting distance of 4 meters at the time points indicated in Tables 8-10. The test will be administered first monocularly (right/left eye) and then binocularly (both eyes). These measures must be performed prior to dilation of the eyes.

BCVA will be measured at following schedule specified in the SoA (Tables 8-10) prior to dilation. Monocular BCVA for both eyes and binocular BCVA tests will be conducted.
The following are needed to conduct the examination:
   a. Examination lane of adequate dimensions to allow testing at required starting distance of 4 meters
   b. Standard chair with a firm back
   C. Set of 3 visual acuity charts (Original Series ETDRS Charts R, 1, and 2, EU Wide ETDRS Charts 1, 2, 3, or PV Number Charts 2750, 2750A, and 2750B)
   d. Retro-Illuminated fluorescent or LED lightbox
   e. Study frame
   f. Study lens set
A VA specifications document, procedure manual, and training materials will be provided to the investigational sites.
The VA examination room and equipment must be validated before any VA examinations are performed.

The same requirements as the BCVA described above apply for LL BCVA. In addition, LLVA will be measured by placing a 2.0-log-unit neutral density filter (Kodak Wratten 2.0 Neutral Density Filter) over the best correction for that eye and having the patient read the normally illuminated ETDRS charts.

Reading speeds will be assessed by MNRead Acuity Charts or Radner Reading Charts at the time points indicated in the SoA. The test will be administered first monocularly (right/left eye) and then binocularly (both eyes). Reading speed testing is only applicable if the charts are available in the local language.

The MNRead acuity cards can be used to measure the reading speed according to the schedule specified in the SoA (Tables 8-10). The detailed information about equipment required, card illumination, viewing distance, test procedure and instruction to patients will be provided to the study sites prior to the start of the study.

The Radner Reading Cards can be used to measure the reading speed if MNRead card is not available. The assessment is to be conducted in each eye separately and then with both eyes open at the schedule specified in the SoA (Tables 8-10). The test consists of 24 short sentences that are highly comparable in terms of number of words, word length, position of words, lexical difficulty, and syntactical complexity. The detailed information about equipment required, card illumination, viewing distance, test procedure and instruction to patients will be provided to the study sites prior to the start of the study.

### Patient Reported Outcomes

The NEI VFQ-25 scores will be assessed as a secondary endpoint.

### Exploratory Efficacy Assessments

Other anatomical and functional outcomes will be measured and analyzed in an exploratory manner.

### GA Lesion Area by SD-OCT

GA area measured by SD-OCT will also be evaluated by masked graders per the Central Reading Manual.

### Mesopic Microperimetry

Mesopic microperimetry will be conducted in a subset of patients who meet the eligibility criteria for microperimetry (microperimetry subpopulation). Microperimetry should be performed prior to eye dilation and any imaging procedure. Two microperimetry tests per eye should be conducted during screening visit and once per eye in the follow up visits.

Microperimetry will evaluate retinal sensitivity which has been shown to be well correlated with anatomical changes in intermediate age-related macular degeneration (iAMD) and GA patients in multiple studies (see Alibhai et al., Int J Retina Vitreous. 2020; 6:16-16; Jones et al., Invest Ophthalmol Vis Sci. 2016; 57(14):6349-6359; Pfau et al., Retina. 2020; 40(1):169-180; Welker et al., Invest Ophthalmol Vis Sci. 2018; 59(4):Amd152-amd159; and Wu et al., Invest Ophthalmol Vis Sci. 2015; 56(3):1546-1552).

A scotoma is an area of reduced sensitivity in the visual field. Mesopic microperimetry evaluates macular functional response and macular sensitivity by quantifying scotomatous points (nonresponding points or “dense” scotomas) in the macula (Csaky et al. (2019), supra). The number of scotomatous points and mean changes of macular sensitivity in total area scanned and in predefined 5 perilesional points around GA lesion in study and fellow eye will be evaluated by the reading center.

### Disease Conversion

Spectral domain-optical coherence tomography (SD-OCT) along with other imaging techniques including FAF, optical coherence tomography angiography (OCTA), fluorescein angiography (FA), and near infrared reflectance (NIR) will be used to evaluate the disease conversion in both eyes, according to classification of AMD (see Table 1). SD-OCT has become an essential imaging technology to evaluate the macula. SD-OCT affords us an opportunity to identify the early stages of the atrophic process before lesions are clinically visible or detected as atrophy by CFP or FAF. In addition, the depth resolved nature of SD-OCT imaging allows us to evaluate tissue layer by layer, which is important because the severity of cellular loss in atrophic disease may vary among layers. SD-OCT has been widely used in recent clinical studies for GA (see Holz et al., Ophthalmology. 2017; 124(4):464-478; and Sadda et al., Ophthalmology. 2018; 125(4):537-548).

Anatomical lesion features from iAMD including high risk drusen, incomplete retinal pigment epithelium and outer retinal atrophy (IRORA), and non-exudative nAMD will be identified from SD-OCT images by masked graders per the Central Reading Manual, the conversion to late AMD including complete retinal pigment epithelium and outer retinal atrophy (cRORA) and exudative nAMD will be tracked over time including:
Conversion from iRORA to cRORA will be assessed at Week 52 and Week 104.
Conversion from high risk drusen to late AMD at Week 52 and Week 104
Conversion from iAMD to late AMD will be assessed at Week 52 and Week 104.

The assessment will be based on standard disease classification definitions provided in Table 1 above.

### Drusen Volume

Evidence from previous study demonstrated that patients with a drusen volume over 0.03 mm³ had an increased risk for developing late AMD compared with those with lower drusen volumes (Abdelfattah, 2016; Folgar, 2016). Drusen volume in the fellow eye with early/iAMD will be measured using SD-OCT images by masked graders per the Central Reading Manual at the time points indicated in Tables 8-10.

### Health Related Quality of Life and Activities of Daily Living Assessments

Health-related QoL will be evaluated using the EuroQoL 5 dimension 5 level (EQ-5D-5L) and National Eye Institute Visual Function 25-item Questionnaire (NEI VFQ-25). Activity of daily living will be assessed using the Lawton Instrumental Activities of Daily Living (IADL) Scale.

Patient reported outcomes will be captured using an electronic device. Validated local language versions of each of the tools will be provided, as needed. All measures should be administered by the Investigator or a qualified site staff, if possible, prior to other study procedures at visits specified in the SoAs.

### National Eye Institute 25-Item Visual Function Questionnaire

The NEI VFQ-25 scores will be analyzed as a secondary endpoint. The NEI VFQ-25 should be administered by the Investigator or a qualified site staff prior to other study procedures at visits specified in the SoAs.

### EuroQoL 5-Dimensions 5-Level

The EuroQoL 5-dimension 5-level questionnaire (EQ-5D-5L) scores will be analyzed as an exploratory endpoint. The EQ-5D-5L should be administered by the Investigator or a qualified site staff prior to other study procedures at visits specified in the SoAs.

### Lawton Instrumental Activities of Daily Living

The Lawton IADL scores will be analyzed as an exploratory endpoint. The change from Baseline will be assessed at its prespecified time points as shown in the SoAs. The IADL should be administered by the Investigator or a qualified site staff prior to other study procedures at visits specified in the SoAs.

### OTHER EMBODIMENTS

This specification has been described with reference to certain embodiments and Examples. However, one of ordinary skill in the art appreciates that various modifications and changes can be made without departing from the scope of the invention as set forth in the claims below. Accordingly, the specification is to be regarded in an illustrative rather than a restrictive sense, and all such modifications are intended to be included within the scope of the claimed invention.

We claim:

1. A method of treatment, wherein the method comprises treating geographic atrophy (GA) secondary to age-related macular degeneration (AMD) in at least one eye in a subject, said treating comprising administering to the subject an effective amount of Compound 1: 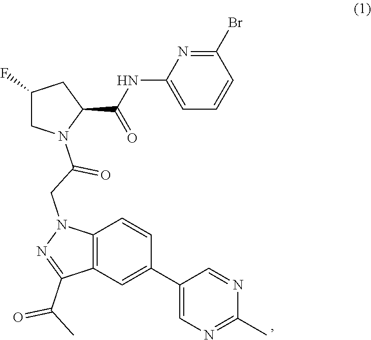 [SMILES: CC(=O)c1nn(CC(=O)N2C[C@H](F)C[C@H]2C(=O)Nc2cccc(Br)n2)c2ccc(-c3cnc(C)nc3)cc12]
or a pharmaceutically acceptable salt thereof, wherein the treatment comprises slowing or reversing a progression of the GA, wherein the slowing the progression of the GA comprises a reduction in a mean rate of increase in a total GA lesion area from baseline in the at least one eye, and the reversing the progression of the GA comprises a mean decrease in the total GA lesion area from baseline in the at least one eye.

2. The method of claim 1, wherein Compound 1 or the pharmaceutically acceptable salt thereof is administered in a dosing regimen of about 50-250 mg twice daily (BID).

3. The method of claim 2, wherein Compound 1 or the pharmaceutically acceptable slat thereof is administered in a dosing regimen of about 100 mg BID.

4. The method of claim 2, wherein Compound 1 or the pharmaceutically acceptable slat thereof is administered in a dosing regimen of about 200 mg BID.

5. The method of claim 1, wherein Compound 1 or the pharmaceutically acceptable salt thereof is administered in a dosing regimen of about 200 mg to about 800 mg once daily (QD).

6. The method of claim 5, wherein Compound 1 or the pharmaceutically acceptable salt thereof is administered in a dosing regimen or about 400 mg QD.

7. The method of any one of claims 1-6, wherein Compound 1 or the pharmaceutically acceptable salt thereof is systemically administered.

8. The method of claim 7, wherein Compound 1 or the pharmaceutically acceptable salt thereof is orally administered.

9. The method of claim 7, wherein Compound 1 or the pharmaceutically acceptable salt thereof is subcutaneously administered.

10. The method of any one of claims 1-6, wherein Compound 1 or the pharmaceutically acceptable salt thereof is ophthalmically administered.

11. The method of any one of claims 1-10, wherein the subject is at least about 60 years of age.

12. The method of any one of claims 1-11, wherein the subject is at least about 65 years of age.

13. The method of any one of claims 1-12, wherein slowing or reversing of the progression of the GA is observed within 104 weeks of treatment.

14. The method of any one of claims 1-12, wherein slowing or reversing of the progression of the GA is observed within 52 weeks of treatment.

15. The method of any one of claims 1-14, wherein the GA secondary to AMD comprises loss of retinal pigment epithelium (RPE) in the eye.

16. The method of any one of claims 1-15, wherein the at least one eye has a GA lesion that is completely visualized on a macula centered color fundus photography (CFP) image and is imaged in its entirety.

17. The method of any one of claims 1-16, wherein the at least one eye has a GA area of 0.35 to 17.76 mm² as measured by fundus autofluorescence (FAF).

18. The method of any one of claims 1-17, wherein the at least one eye does not have exudative neovascular age-related macular degeneration (nAMD).

19. The method of any one of claims 1-17, wherein the subject has nAMD in the at least one eye.

20. The method of any one of claims 1-19, wherein the slowing of the progression of the GA further comprises a reduction in a mean rate of increase in a square root of a total GA lesion area from baseline as measured by FAF in (a) the at least one eye; (b) a fellow eye if GA is present at the fellow eye at baseline; or (c) both the at least one eye and the fellow eye, regardless of baseline GA status of the at least one eye and the fellow eye; and the reversing the progression of the GA further comprises a mean decrease in the square root of the total GA lesion area from baseline as measured by FAF in (a) the at least one eye; (b) the fellow eye if GA is present at the fellow eye at baseline; or (c) both the at least one eye and the fellow eye, regardless of baseline GA status of the at least one eye and the fellow eye.

21. The method of any one of claims 1-20, wherein the reduction in the mean rate of increase or decrease in the total GA lesion area in the at least one eye is measured by FAF.

22. The method of any one of claims 1-21, wherein the slowing of the progression of the GA further comprises a reduction in a mean rate of increase in a total GA lesion area from baseline as measured by FAF in (a) the at least one eye; (b) a fellow eye if GA is present at the fellow eye at baseline; or (c) both the at least one eye and the fellow eye, regardless of baseline GA status of the at least one eye and the fellow eye; and the reversing the progression of the GA further comprises a mean decrease in the total GA lesion area from baseline as measured by FAF in (a) the at least one eye; (b) the fellow eye if GA is present at the fellow eye at baseline; or (c) both the at least one eye and the fellow eye, regardless of baseline GA status of the at least one eye and the fellow eye.

23. The method of any one of claims 1-22, wherein the slowing of the progression of the GA further comprises a reduction in a mean increase in a total GA lesion area from baseline as measured by FAF in (a) the at least one eye; (b) a fellow eye if GA is present at the fellow eye at baseline; or (c) both the at least one eye and the fellow eye, regardless of baseline GA status of the at least one eye and the fellow eye; and the reversing the progression of the GA further comprises a mean decrease in the total GA lesion area from baseline as measured by FAF in (a) the at least one eye; (b) the fellow eye if GA is present at the fellow eye at baseline; or (c) both the at least one eye and the fellow eye, regardless of baseline GA status.

24. The method of any one of claims 1-23, wherein the slowing of the progression of the GA further comprises a reduction in a mean increase in a square root of a total GA lesion area from baseline as measured by FAF in (a) the at least one eye; (b) a fellow eye if GA is present at the fellow eye at baseline; or (c) both the at least one eye and the fellow eye, regardless of baseline GA status of the at least one eye and the fellow eye; and the reducing the GA further comprises a mean decrease in the square root of the total GA lesion area from baseline as measured by FAF in (a) the at least one eye; (b) the fellow eye if GA is present at the fellow eye at baseline; or (c) both the at least one eye and the fellow eye, regardless of baseline GA status of the at least one eye and the fellow eye.

25. The method of any one of claims 1-23, wherein the treatment of GA secondary to AMD further comprises an improvement in a monocular best-corrected visual acuity (BCVA) score in the at least one eye from baseline as assessed by Early Treatment Diabetic Retinopathy Study (ETDRS) chart.

26. The method of any one of claims 1-24, wherein the treatment of GA secondary to AMD further comprises an improvement in a monocular BCVA score in a fellow eye from baseline assessed by ETDRS chart.

27. The method of any one of claims 1-26, wherein the treatment of GA secondary to AMD further comprises an improvement in a monocular low luminance visual acuity (LLVA) score in the at least one eye from baseline as assessed by ETDRS chart.

28. The method of any one of claims 1-27, wherein the treatment of GA secondary to AMD further comprises an improvement in a monocular LLVA score in a fellow eye from baseline assessed by ETDRS chart.

29. The method of any one of claims 1-28, wherein the treatment of GA secondary to AMD further comprises an improvement in a low luminance deficit (LLD) score in the at least one eye from baseline.

30. The method of any one of claims 1-29, wherein the treatment of GA secondary to AMD further comprises an improvement in an LLD score in a fellow eye from baseline.

31. The method of any one of claims 1-30, wherein the treatment of GA secondary to AMD further comprises an improvement in a National Eye Institute Visual Function Questionnaire, 25-item versions (NEI VFQ-25) score from baseline.

32. The method of any one of claims 1-31, wherein the treatment of GA secondary to AMD further comprises by an improvement in an EQ-5D-5L score in one or more of mobility, usual activities, self-care, pain or discomfort, and anxiety/depression.

33. The method of any one of claims 1-32, wherein the treatment of GA secondary to AMD further comprises an improvement in a Lawson Instrumental Activities of Daily Living (IADL) score from baseline.

34. The method of any one of claims 1-33, wherein the treatment of GA secondary to AMD further comprises an improvement in monocular reading speed as assessed by a Minnesota Low-Vision Reading Test (MNRead) or with Radner Reading Charts.

35. The method of claim 34, wherein the improvement in monocular reading speed is assessed in the at least one eye.

36. The method of claim 34, wherein the improvement in monocular reading speed is assessed in a fellow eye.

37. The method of any one of claims 1-36, wherein the treatment of GA secondary to AMD further comprises an improvement in binocular reading speed as assessed by MNRead or with Radner Reading Cards.

38. The method of any one of claims 25-37, where the mean improvement from baseline determined based on a comparison with a control.

39. The method of claim 38, wherein the control is untreated subjects.

40. The method of claim 38, wherein the control is placebo-treated subjects.

41. The method of any one of claims 1-40, wherein the slowing of the progression of the GA further comprises a reduction in a mean rate of increase in ellipsoid zone loss area within and outside GA in a predetermined area centered on the fovea from baseline as measured by spectral-domain optical coherence tomography (SD-OCT) in (a) the at least one eye, (b) a fellow eye if GA is present at the fellow eye at baseline, or (c) both eyes; and the reversing of the progression of the GA further comprises a decrease in ellipsoid zone loss area within and outside GA in a predetermined area centered on the fovea from baseline as measured by SD-OCT.

42. The method of any one of claims 1-41, wherein the subject has incomplete retinal pigment epithelium and outer retinal atrophy (IRORA) in a fellow eye, and the treatment of the GA secondary to AMD further comprises reducing the risk of the IRORA in the fellow eye converting into complete retinal pigment epithelium and outer retinal atrophy (cRORA) as determined by SD-OCT.

43. The method of any one of claims 1-42, wherein the subject has incomplete iRORA in the at least one eye, and the treatment of the GA secondary to AMD further comprises reducing the risk of the iRORA in the at least one eye converting into cRORA as determined by SD-OCT.

44. The method of any one of claims 1-43, wherein the subject has a high-risk drusen in a fellow eye, and the treatment of the GA secondary to AMD further comprises reducing the risk of the high-risk drusen in the fellow eye converting into late AMD as determined by SD-OCT.

45. The method of any one of claims 1-44, wherein the subject has intermediate AMD (iAMD) in a fellow eye, and the treatment of the GA secondary to AMD further comprises reducing the risk of the iAMD converting into late AMD as determined by SD-OCT.

46. The method of any one of claims 1-45, wherein the subject has iAMD in a fellow eye, and the treatment of the GA secondary to AMD further comprises reducing the risk of the iAMD converting into late AMD as determined by SD-OCT.

47. The method of any one of claims 1-46, wherein the subject does not have GA in a fellow eye, and the treatment of GA secondary to AMD further comprises a reduction in a mean increase in drusen surface volume in (a) the fellow eye and/or (b) both eyes combined; or a mean decrease in drusen volume in (a) the fellow eye and/or (b) both eyes combined.

48. The method of any one of claims 1-47, wherein the slowing of the progression of the GA further comprises a reduction in a mean increase in a total GA lesion area as measured by SD-OCT in (a) the at least one eye; (b) a fellow eye if GA is present at the fellow eye at baseline; or (c) both eyes combined, regardless of baseline GA status in the at least one eye and the fellow eye; and the reversing of the progression of the GA further comprises a mean decrease in total GA lesion area as measured by SD-OCT in (a) the at least one eye; (b) a fellow eye if GA is present at the fellow eye at baseline; or (c) both eyes combined, regardless of baseline GA status in the at least one eye and the fellow eye.

49. The method of any one of claims 1-48, wherein the GA secondary to AMD comprises extrafoveal GA, and the subject can detect a fixation target.

50. The method of claim 49, wherein the subject requires ≤30 minutes to complete a microperimetry test for each eye, and where the reliability test ratio is ≤20%.

51. The method of claim 49 or 50, wherein the slowing of the progression of the GA secondary to AMD further comprises a reduction in a decrease in macular sensitivity from baseline as assessed by mesopic microperimetry, and the reversing the progression of the GA further comprises an increase in macular sensitivity from baseline as assessed by mesopic microperimetry.

52. The method of any one of claims 49-51, wherein the slowing of the progression of the GA further comprises a reduction in an increase in a number of scotomatous points from baseline as assessed by mesopic microperimetry, and the reversing of the progression of the GA further comprises a decrease in the number of scotomatous points from baseline as assessed by mesopic microperimetry.

53. The method of any one of claims 1-52, wherein the GA comprises a GA lesion >1 μm outside of the foveal center outside of a foveal center.

54. The method of any one of claims 1-53, wherein the slowing or reversing of the progression of the GA or the reducing the GA is determined based on a comparison between the subject and a control.

55. The method of claim 54, wherein the control is untreated subjects.

56. The method of claim 55, wherein the control is placebo-treated subjects.

57. The method of any one of claims 1-56, wherein the slowing or reversing of the progression of the GA or the reducing the GA is determined based on a comparison between the at least one eye and a fellow eye.

58. The method of any one of claims 1-57, wherein the eye with GA secondary to AMD has not received an intravitreal anti vascular endothelial growth factor (VEGF) injection for intraocular vascular disease prior to receiving treatment.

59. The method of any one of claims 1-58, wherein the eye with GA secondary to AMD has not received an intravitreal anti VEGF injection prior to receiving treatment.

60. A method of treatment, the method comprising treating intermediate AMD (iAMD) in at least one eye in a subject, said treating comprising administering to the subject an effective amount of Compound 1: 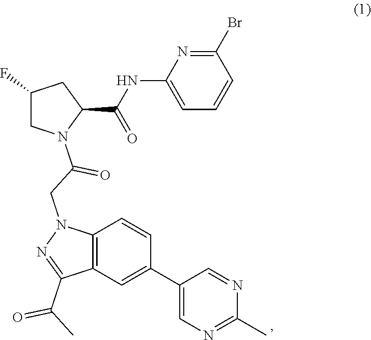 [SMILES: CC(=O)c1nn(CC(=O)N2C[C@H](F)C[C@H]2C(=O)Nc2cccc(Br)n2)c2ccc(-c3cnc(C)nc3)cc12]
or a pharmaceutically acceptable salt thereof, wherein the treatment comprises reducing the risk of the iAMD converting into late AMD as measured by SD-OCT.

61. The method of claim 60, wherein the subject has iAMD in both eyes.

62. The method of claim 60 or 61, wherein the iAMD comprises iRORA, and the late AMD comprises cRORA.

63. The method of claim 60 or 61, wherein the iAMD comprises a high-risk drusen.

64. The method of any one of claims 60-63, wherein Compound 1 or the pharmaceutically acceptable salt thereof is administered in a dosing regimen of about 50-250 mg BID.

65. The method of claim 64, wherein Compound 1 or the pharmaceutically acceptable slat thereof is administered in a dosing regimen of about 100 mg BID.

66. The method of claim 64, wherein Compound 1 or the pharmaceutically acceptable slat thereof is administered in a dosing regimen of about 200 mg BID.

67. The method of any one of claims 60-63, wherein Compound 1 or the pharmaceutically acceptable salt thereof is administered in a dosing regimen of about 200 mg to about 800 mg QD.

68. The method of claim 67, wherein Compound 1 or the pharmaceutically acceptable salt thereof is administered in a dosing regimen or about 400 mg QD.

69. The method of any one of claims 60-68, wherein Compound 1 or the pharmaceutically acceptable salt thereof is systemically administered.

70. The method of claim 69, wherein Compound 1 or the pharmaceutically acceptable salt thereof is orally administered.

71. The method of claim 69, wherein Compound 1 or the pharmaceutically acceptable salt thereof is subcutaneously administered.

72. The method of any one of claims 60-68, wherein Compound 1 or the pharmaceutically acceptable salt thereof is ophthalmically administered.

73. The method of any one of claims 60-72, wherein the subject is at least about 60 years of age.

74. The method of any one of claims 60-72, wherein the subject is at least about 65 years of age.

75. The method of any one of claims 60-74, wherein the iAMD does not convert into late AMD within a 104-week treatment period.

76. The method of any one of claims 60-75, wherein the iAMD does not convert into late AMD within a 54-week treatment period.

77. The method of any one of claims 1-76, wherein the subject has a glomerular filtration rate ≥30 mL/min/1.73 m².

78. The method of any one of claims 1-77, wherein the subject has been vaccinated against meningococcal infections within 3 years prior to receiving treatment.

79. The method of any one of claims 1-78, wherein the subject has been vaccinated against meningococcal infection within less than 2 weeks prior to receiving treatment, and is further administered a prophylactic antibiotic until two weeks after vaccination.

80. The method of any one of claims 1-79, wherein the at least one eye has a visual acuity (VA) score of 84 to 4 letters or 20/20 to 20/800 as determined using Early Treatment Diabetic Retinopathy Study (ETDRS) charts at starting distance of 4 meters.

81. The method of any one of claims 1-79, wherein the at least one eye has a visual acuity (VA) score of 84 to 24 letters or 20/20 to 20/320 as determined using ETDRS charts at starting distance of 4 meters.

82. The method of any one of claims 1-81, wherein the subject has not received any complement, stem cell, or gene therapy for any ophthalmological condition prior to receiving treatment.

83. The method of any one of claims 1-81, wherein the subject has not received any stem cell or gene therapy for any ophthalmological condition prior to receiving treatment.

84. The method of any one of claims 1-82, wherein the subject has not received treatment for drusen, nascent geographic atrophy, or GA via any route of administration in either eye.

85. The method of any one of claims 1-84, wherein the subject has not received laser photocoagulation therapy for nAMD, diametric macular edema, retinal vein occlusion, and/or proliferative diabetic retinopathy in either eye.

86. The method of any one of claims 1-85, wherein the subject has not received laser photocoagulation therapy for nAMD, diametric macular edema, retinal vein occlusion, and/or proliferative diabetic retinopathy in the study eye.

87. The method of any one of claims 1-86, wherein the subject has not received photodynamic therapy or transpupillary thermotherapy in the study eye.

88. The method of any one of claims 1-87, wherein the subject has not received external bean radiation therapy and/or any other irradiation to the study and respective orbit, head, and/or neck,

89. The method of any one of claims 1-88, wherein the subject has not received photodynamic therapy in either eye; internal beam radiation therapy and/or any other irradiation to the eye, orbit, head, and or neck; or transpupillary thermotherapy in either eye.

90. The method of any one of claims 1-89, wherein the subject has not received intravitreal delivery of steroid, anti-complement, or device implantation in either eye, provided that the intravitreal steroid delivery is not for cystoid macular edema after cataract ≥3 months.

91. The method of any one of claims 1-90, wherein the subject has not received intravitreal delivery of steroid or device implantation in either eye, provided that the intravitreal steroid delivery is not for cystoid macular edema after cataract ≥3 months.

92. The method of any one of claims 1-91, wherein the subject does not have a history of recurrent infectious or inflammatory eye disease in either eye.

93. The method of any one of claims 1-92, wherein the subject does not have a history of retinal detachment or macular hole in either eye.

94. The method of any one of claims 1-93, wherein the subject does not have a history of glaucoma-filtering surgery in either eye.

95. The method of any one of claims 1-94, wherein the subject does not have a history of corneal transplantation in either eye.

96. The method of any one of claims 1-95, wherein the subject does not have a history of vitrectomy, submacular surgery, or any surgical intervention for AMD in either eye.

97. The method of any one of claims 1-96, wherein the subject has not received intraocular surgery within 3 months prior to treatment.

98. The method of any one of claims 1-97, wherein the subject does not have a known or suspected complement deficiency.

99. The method of any one of claims 1-98, wherein the subject does not have a history of N meningitidis infection.

100. The method of any one of claims 1-99, wherein the subject does not have or exhibit signs of an active bacterial, viral, or other infection; does not have a body temperature of >38° C. on two consecutive days, and does not have any febrile illness within 14 days prior to receiving treatment.

101. The method of any one of claims 1-100, wherein the subject does not have a history of a malignant disease within 5 years prior to treatment or an ongoing malignant disease, provided that the malignant disease is not a basal cell or squamous cell carcinoma of the skin that has been completely excised and/or cured.

102. The method of any one of claims 1-101, wherein the subject does not have an ALT, aspartate aminotransferase (AST), alkaline phosphatase (ALP), or direct bilirubin >2×upper limit of normal (ULN).

103. The method of any one of claims 1-101, wherein the subject has a direct bilirubin level >2×ULN, provided that the subject has Gilbert's Syndrome.

104. The method of any one of claims 1-103, wherein the subject is not exhibiting signs of hepatobiliary cholestasis.

105. The method of any one of claims 1-104, wherein the subject is not exhibiting signs of a hepatitis B viral infection with negative surface antibodies.

106. The method of any one of claims 1-105, wherein the subject is not exhibiting signs of a hepatitis C viral infection.

107. The method of any one of claims 1-106, wherein the subject is not exhibiting signs of a human immunodeficiency viral infection.

108. Use of Compound 1: 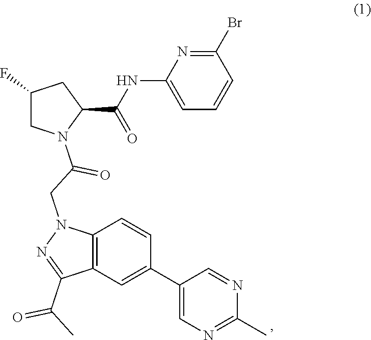 [SMILES: CC(=O)c1nn(CC(=O)N2C[C@H](F)C[C@H]2C(=O)Nc2cccc(Br)n2)c2ccc(-c3cnc(C)nc3)cc12]
or a pharmaceutically acceptable salt thereof in the manufacture of a medicament for use in the method of any one of claims 1-107.

109. A compound for use in the method of any one of claims 1-107, wherein the compound is Compound 1: 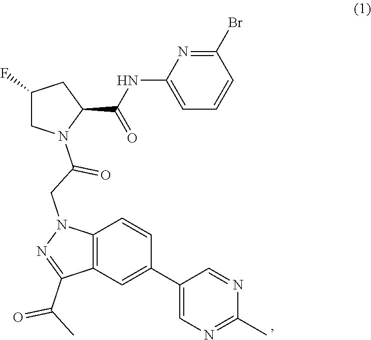 [SMILES: CC(=O)c1nn(CC(=O)N2C[C@H](F)C[C@H]2C(=O)Nc2cccc(Br)n2)c2ccc(-c3cnc(C)nc3)cc12]
or a pharmaceutically acceptable salt thereof.

110. A kit for treating GA secondary to AMID and/or IAMD in at least one eye in a subject, comprising:
(a) a dose of Compound 1: 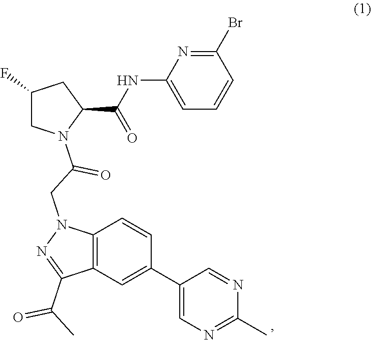 [SMILES: CC(=O)c1nn(CC(=O)N2C[C@H](F)C[C@H]2C(=O)Nc2cccc(Br)n2)c2ccc(-c3cnc(C)nc3)cc12]
or a pharmaceutically acceptable salt thereof; and
(b) instructions for using Compound 1 or the pharmaceutically acceptable salt thereof according to the method of any one of claims 1-107.
